(19) United States
(12) Patent Application Publication
(10) Pub. No.: US 2026/0314978 A1
(43) Pub. Date: Oct. 15, 2026

(54) METHOD AND APPARATUS FOR MONITORING CONDUCTION SYSTEM PACING

(71) Applicant: Medtronic, Inc., Minneapolis, MN (US)

(72) Inventors: Jian CAO, Shoreview, MN (US); Xiaohong ZHOU, Woodbury, MN (US); Deborah Ann JAYE, Lino Lakes, MN (US); Wade M. DEMMER, Coon Rapids, MN (US); Hongyang LU, Shanghai (CN); Tianyi SHI, Shanghai (CN); Manjunathan YELLAPPAN PADUA, Shoreview, MN (US); Kiran Kumar YANDRA, Mounds View, MN (US)

(21) Appl. No.: 18/996,917

(22) PCT Filed: Jul. 20, 2022
(86) PCT No.: PCT/CN2022/106665

§ 371 Date: Jan. 17, 2025

(51) Int. Cl. A61B 5/29; A61B 5/00; A61B 5/0245; A61B 5/339; A61B 5/35

(52) U.S. Cl. CPC A61B 5/29; A61B 5/0031; A61B 5/0245; A61B 5/339; A61B 5/35; A61B 5/4836; A61B 5/6869

(57) ABSTRACT

A medical device is configured to obtain, by processing circuitry of the medical device, at least a first electrocardiogram (ECG) signal. The processing circuitry may determine a ventricular activation time using the first ECG signal for each one of multiple pacing pulses and detect a threshold difference between a first ventricular activation time associated with a first pacing pulse of the plurality of pacing pulses and a second ventricular activation time associated with a second pacing pulse of the plurality of pacing pulses. The medical device may be further configured to display the determined ventricular activation times.

### TECHNICAL FIELD

This disclosure relates to a medical device and method for monitoring conduction system pacing of a patient's heart.

### BACKGROUND

During normal sinus rhythm (NSR), the heartbeat is regulated by electrical signals produced by the sino-atrial (SA) node located in the right atrial wall. Each depolarization signal produced by the SA node spreads across the atria, causing the depolarization and contraction of the atria, and arrives at the atrioventricular (AV) node. The AV node responds by propagating a depolarization signal through the bundle of His of the atrioventricular septum and thereafter to the bundle branches and the Purkinje muscle fibers of the right and left ventricles, sometimes referred to as the “His-Purkinje system.”

Patients with a conduction system abnormality, e.g., poor AV node conduction, poor SA node function, or other conduction abnormalities, may receive a pacemaker to restore a more normal heart rhythm and heart chamber synchrony. Ventricular pacing may be performed to maintain the ventricular rate in a patient having atrioventricular conduction abnormalities. A single chamber ventricular pacemaker may be coupled to a transvenous ventricular lead carrying electrodes placed in the right ventricle (RV), e.g., in the right ventricular apex. The pacemaker itself is generally implanted in a subcutaneous pocket with the transvenous ventricular lead tunneled to the subcutaneous pocket. Intracardiac pacemakers have been introduced or proposed for implantation entirely within a patient's heart, eliminating the need for transvenous leads. An intracardiac pacemaker may provide sensing and pacing from within a chamber of the patient's heart, e.g., from within the right ventricle in a patient having AV conduction block.

Dual chamber pacemakers are available which include a transvenous atrial lead carrying electrodes which are placed in the right atrium and a transvenous ventricular lead carrying electrodes that are placed in the right ventricle via the right atrium. A dual chamber pacemaker senses atrial electrical signals and ventricular electrical signals and can provide both atrial pacing and ventricular pacing as needed to promote a normal atrial and ventricular rhythm and promote AV synchrony when SA and/or AV node or other conduction abnormalities are present.

Ventricular pacing via electrodes at or near the right ventricular apex has been found to be associated with increased risk of atrial fibrillation and heart failure. Alternative pacing sites have been investigated or proposed, such as pacing of the His bundle or left bundle branch. Ventricular pacing along the His-Purkinje conduction system has been proposed to provide a more physiologic form of ventricular pacing because pacing-evoked depolarizations can be conducted along the heart's natural conduction system. Pacing the ventricles via the His bundle or left bundle branch, for example, allows recruitment along the heart's natural conduction system, including the bundle branches and the Purkinje fibers, and is hypothesized to promote more physiologically normal cardiac activation than other pacing sites, such as at the ventricular apex.

### SUMMARY

The techniques of this disclosure generally relate to a medical device system for monitoring pacing of the His-Purkinje conduction system, also referred to herein as the “conduction system,” of a patient's heart Conduction system pacing (CSP) may be delivered by placing at least one electrode along or in the vicinity of the His-Purkinje conduction system, which may be along the His bundle and/or along or in the vicinity of one or both of the left bundle branch (LBB) and/or right bundle branch (RBB). In various examples, a medical device operating according to methods disclosed herein receives input signals from electrodes, e.g., surface electrocardiogram (ECG) electrodes, determines a differential ECG signal from the signal inputs, and determines a ventricular activation time from the differential ECG signal, which may be a unipolar or bipolar ECG signal. The ventricular activation time (VAT) may be determined from a delivered CSP pulse to a fiducial point of an ECG signal. The medical device can be configured to display a visual representation of the VAT in a graphical user interface (GUI). The VAT may be determined for multiple cardiac cycles during which a CSP pulse is delivered by the medical device or by a second medical device, such as an implantable pacemaker.

When a CSP pulse captures at least a portion of the conduction system, a sudden change, e.g., a decrease, in the VAT can be expected to occur. The VATs determined for multiple cardiac cycles can be presented to a clinician or user in a visual display with a conspicuous indicator, e.g., a change in the formatting of the VATs or other visual and/or audible notification, presented to a user to notify the user of a detected change in determined VATs. A change in the determined VATs can indicate a change in capture of the conduction system (e.g., successful capture or loss of capture). The techniques disclosed herein for determining a change in VAT and/or other metrics determined from one or more ECG signals can provide a user guidance in positioning at least one electrode during an implantation procedure and/or in adjusting CSP parameters for achieving CSP capture.

In one example, the disclosure provides a medical device including a processor configured to obtain one or more ECG signals. The processor may be configured to, for each of a plurality of pacing pulses, determine a ventricular activation time using a first ECG signal of the one or more ECG signals, determine a difference between the ventricular activation time and a previously determined ventricular activation time; and determine when the difference meets a threshold difference. The medical device may include a display unit in communication with the processor. The display unit can be configured to display at least one of the one or more ECG signals, display at least one of the ventricular activation times or the determined differences, and display a conspicuous indicator in response to determining that the threshold difference is met.

In another example, the disclosure provides a method including obtaining one or more ECG signals and, for each of a plurality of pacing pulses, determining a ventricular activation time using a first ECG signal of the one or more ECG signals, determining a difference between the ventricular activation time and a previously determined ventricular activation time; and determining when the difference meets a threshold difference. The method can further include displaying at least one of the one or more ECG signals, displaying at least one of the determined ventricular activation times or the determined differences, and displaying a conspicuous indicator in response to determining that the threshold difference is met.

In another example, the disclosure provides a non-transitory computer readable medium storing instructions which, when executed by processing circuitry of a medical device, cause the medical device to obtain one or more ECG signals. For each of a plurality of pacing pulses, the instructions cause the medical device to determine a ventricular activation time using a first ECG signal of the one or more ECG signals, determine a difference between the ventricular activation time and a previously determined ventricular activation time and determine when the difference meets a threshold difference. The instructions further cause the medical device to display at least one of the one or more ECG signals, display at least one of the determined ventricular activation times or the determined differences, and display a conspicuous indicator in response to determining that the threshold difference is met.

Further disclosed herein is the subject matter of the following examples:

Example 1. A medical device including a processor configured to obtain one or more ECG signals. The processor is further configured to, for each of a plurality of pacing pulses, determine a ventricular activation time using a first ECG signal of the one or more ECG signals, determine a difference between the ventricular activation time and a previously determined ventricular activation time, and determine that the difference meets a threshold difference. The medical device further includes a display unit in communication with the processor and configured to display at least one of the one or more ECG signals, display at least one of the ventricular activation times or the determined differences and display a conspicuous indicator in response to determining that the threshold difference is met.

Example 2. The medical device of example 1 wherein the processor is further configured to obtain the first ECG signal by receiving four input signals from each of a reference electrode and three chest electrodes, the three chest electrodes including a left chest electrode and a right chest electrode, determining a central terminal signal from the four input signals, and determining a first unipolar ECG signal as a difference between an input signal from the left chest electrode and the central terminal signal.

Example 3. The medical device of example 2 wherein the processor is further configured to obtain the first ECG signal by determining a second unipolar ECG signal from the input signals and determining the first ECG signal as a bipolar ECG signal by determining a difference between the first unipolar ECG signal and the second unipolar ECG signal.

Example 4. The medical device of example 2 wherein the processor is further configured to obtain the one or more ECG signals by determining a second unipolar ECG signal as a difference between an input signal from the right chest electrode and the central terminal signal and the display unit is further configured to display a plurality of cardiac cycles of the second unipolar ECG signal corresponding to at least a portion of the plurality of pacing pulses and aligned in time with respective ventricular activation times determined for at least the portion of the plurality of pacing pulses.

Example 5. The medical device of claim 1, 2, 3 or 4 wherein the processor and the display unit are further configured to simultaneously display a plurality of cardiac cycles of at least one of the obtained ECG signal(s) and a plurality of the determined ventricular activation times, wherein the plurality of cardiac cycles of the at least one of the obtained ECG signal(s) and the plurality of ventricular activation times correspond to at least a portion of the plurality of pacing pulses.

Example 6. The medical device of example 1, 2, 3, or 4 wherein the processor and the display unit are further configured to consecutively display each one a plurality of cardiac cycles of at least one of the obtained ECG signal(s), each one of the plurality of cardiac cycles being displayed individually one at a time with a respective determined ventricular activation time, the plurality of cardiac cycles corresponding to at least a portion of the plurality of pacing pulses.

Example 7. The medical device of example 1 wherein the processor is further configured to determine each of the ventricular activation times by identifying a fiducial point of the first ECG signal following a respective one of the plurality of pacing pulses; and determining the ventricular activation time as a time interval from the respective one of the plurality of pacing pulses to the fiducial point. The display unit being further configured to display, with the determined ventricular activation times, the first ECG signal and a visual marker of at least one of the identified fiducial points of the first ECG signal.

Example 8. The medical device of example 1 wherein the processor is further configured to determine when a ventricular activation time of the determined ventricular activation times is greater than a threshold ventricular activation time. The display unit being further configured to display each of the ventricular activation times that are determined to be greater than the threshold ventricular activation time according to a first format and each of the ventricular activation times that are less than the threshold ventricular activation time according to a second format different than the first format.

Example 9. The medical device of example I wherein the processor is further configured to obtain the one or more ECG signals by receiving a plurality of input signals from at least a right chest electrode, a left chest electrode and a reference electrode, the one or more ECG signals comprising the first ECG signal associated with the left chest electrode and a second ECG signal associated with the right chest electrode. The processor being further configured to, for at least one of the plurality of pacing pulses, determine a QRS width using the second ECG signal. The display unit being further configured to display the determined QRS width.

Example 10. The medical device of example 1 wherein the processor is further configured to obtain the one or more ECG signals by receiving a plurality of input signals from at least a right chest electrode, a left chest electrode and a reference electrode, the plurality of ECG signals comprising the first ECG signal associated with the left chest electrode and a second ECG signal associated with the right chest electrode. The processor being further configured to, for at least one of the plurality of pacing pulses, determine at least one of a QRS area using the second ECG signal, a peak interval between a maximum peak of the first ECG signal and the second ECG signal, or a QRS morphology metric. The display unit can be further configured to display at least one of the QRS area, peak interval, or QRS morphology metric.

Example 11. The medical device of example 1, wherein the display unit is further configured to display the conspicuous indicator by at least one of: generating a visual notification, generating an audible notification, adjusting a format of the determined ventricular activation time to be different than a previously displayed ventricular activation time, and/or adjusting a background of the determined ventricular activation time.

Example 12. The medical device of example 1, further comprising a pulse generator configured to deliver the plurality of pacing pulses via a conduction system pacing electrode.

Example 13. The medical device of example 1, further comprising a telemetry circuit configured to receive a conduction system pacing pulse marker signal from an implantable medical device configured to deliver the plurality of pacing pulses as conduction system pacing pulses.

Example 14. The medical device of example 13, wherein the telemetry circuit is further configured to receive a cardiac electrogram signal from the implantable medical device configured to deliver the plurality of pacing pulses as conduction system pacing pulses. The display unit being further configured to display the cardiac electrogram signal.

Example 15. A method including obtaining one or more ECG signals and, for each of a plurality of pacing pulses, determining a ventricular activation time using a first ECG signal of the one or more ECG signals, determining a difference between the ventricular activation time and a previously determined ventricular activation time, and determining when the difference meets a threshold difference. The method can further include displaying at least one of the one or more ECG signals, displaying at least one of the determined ventricular activation times or the determined differences, and displaying a conspicuous indicator in response to determining that the threshold difference is met.

Example 16. The method of example 15 wherein obtaining the first ECG signal includes receiving four input signals from each of a reference electrode and three chest electrodes, the three chest electrodes including a left chest electrode and a right chest electrode, determining a central terminal signal from the four input signals and determining a first unipolar ECG signal as a difference between an input signal from the left chest electrode and the central terminal signal.

Example 17. The method of example 16 wherein obtaining the first ECG signal further includes determining a second unipolar ECG signal from the input signals and determining the first ECG signal as a bipolar ECG signal by determining a difference between the first unipolar ECG signal and the second unipolar ECG signal.

Example 18. The method of claim 16 further including obtaining the one or more ECG signals by determining a second unipolar ECG signal as a difference between an input signal from the right chest electrode and the central terminal signal and displaying a plurality of cardiac cycles of the second unipolar ECG signal corresponding to at least a portion of the plurality of pacing pulses and aligned in time with respective ventricular activation times determined for at least the portion of the plurality of pacing pulses.

Example 19. The method of example 15, 16, 17 or 18 further including simultaneously displaying a plurality of cardiac cycles of at least one of the obtained ECG signal(s) and a plurality of the determined ventricular activation times, wherein the plurality of cardiac cycles of the at least one of the obtained ECG signal(s) and the plurality of ventricular activation times correspond to at least a portion of the plurality of pacing pulses.

Example 20. The medical device of example 15, 16, 17 or 18, further comprising consecutively displaying each one of a plurality of cardiac cycles of at least one of the obtained ECG signal(s), each one of the plurality of cardiac cycles being displayed individually one at a time with a respective determined ventricular activation time, the plurality of cardiac cycles corresponding to at least a portion of the plurality of pacing pulses.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram of a medical device system capable of sensing and analyzing cardiac electrical signals and displaying a GUI including VATs determined by a processor receiving the cardiac electrical signals according to some examples.

FIG. 2 is a conceptual diagram of pacing leads coupled to an implantable medical device (IMD) capable of pacing a patient's heart and sensing cardiac electrical signals via the pacing leads.

FIG. 3 is a conceptual diagram of an IMD coupled to a CSP lead advanced to an alternative location within the heart for delivering CSP pulses and sensing cardiac electrical signals.

FIG. 4 is a conceptual diagram of a leadless pacemaker positioned within the right atrium for providing CSP according to another example.

FIG. 5 is a conceptual diagram of the leadless pacemaker of FIG. 4 shown implanted in an alternative location for CSP.

FIG. 6 is a schematic diagram of circuitry that may be enclosed within an IMD configured to sense cardiac electrical signals and perform CSP.

FIG. 7 is a flow chart of a method for processing and analyzing ECG signals and generating a GUI for display by a medical device according to some examples.

FIG. 8 is a conceptual diagram of processing circuitry that may be included in a medical device for obtaining differential unipolar and bipolar ECG signals according to some examples.

FIG. 9 is an example of a GUI that may be displayed on display unit of a medical device according to one example.

FIG. 10 is a conceptual diagram of two different screenshots that may be displayed at different times in a GUI by a display unit, in cooperation with processing circuitry of a medical device, according to another example.

FIG. 11 is a flow chart of a method for displaying data relating to CSP in a GUI by a medical device according to another example.

FIG. 12 is a diagram of a GUI that may be cooperatively generated by a processor and display unit of a medical device for display on the display unit according to another example.

FIG. 13 is a conceptual diagram of two different screenshots that may be displayed at different times in a GUI by a display unit of a medical device according to another example.

### DETAILED DESCRIPTION

A medical device is described herein for receiving, processing and analyzing at least one ECG signal for monitoring for capture of at least a portion of the conduction system and the resultant improvement in electrical synchrony of the ventricles. The ECG signal analysis can be performed by processing circuitry of a medical device that is configured to receive electrical signals from chest electrodes positioned on a patient during implantation of an implantable pacing device configured to deliver CSP. The ECG signal analysis can be performed during patient follow-ups and during capture threshold tests or any other time when confirmation of CSP capture and/or an improvement in ventricular electrical synchrony due to CSP is desired.

As used herein, the term “CSP” refers to delivery of one or more pacing pulses generated for delivery in the vicinity of a portion of the His-Purkinje conduction system of a heart. A CSP pulse may or may not capture the conduction system depending on the cathode and anode locations of a CSP electrode vector relative to the conduction system pacing site, the delivered pacing pulse energy and other factors. Complete or partial His bundle capture, complete or partial LBB capture and/or complete or partial right bundle branch (RBB) capture are examples of capture of at least a portion of the conduction system. Capture of at least a portion of the conduction system is achieved when the pacing pulse energy delivered in a pacing pulse causes depolarization of tissue of the conduction system. The pacing-evoked depolarization arising at the pacing site can be propagated along the conduction system to the ventricular myocardium to cause depolarization of the ventricular myocardium and subsequent, coordinated ventricular contraction.

The medical device and techniques disclosed herein provide various improvements in a medical device system configured to generate and display various parameters determined from one or more ECG signals that a user may rely on when monitoring and assessing CSP. The techniques disclosed herein improve the function of a medical device in providing visual representations of CSP data useful in guiding a pacing electrode implant procedure, testing for capture of the conduction system, and monitoring for improvement in electrical synchrony achieved by CSP.

The techniques disclosed herein therefore provide improvements in the computer-related field of cardiac monitoring and cardiac therapy delivery. By providing a medical device system capable of displaying a GUI according to the techniques herein, the complexity and likelihood of human error in positioning a pacing electrode at a CSP site and/or selecting CSP parameters for achieving capture of at least a portion of the conduction system is reduced. The clinical benefit of CSP to the patient can be improved by the disclosed techniques by simplifying the process of confirming CSP capture and/or improvement in ventricular electrical synchrony during CSP. The techniques disclosed herein may enable a pacing electrode, which may be a lead-based or housing-based electrode as described in the examples below, to be positioned at a pacing site along the heart's native conduction system for achieving conduction system capture with a high degree of confidence in a manner that is simplified, flexible, and patient-specific. The techniques disclosed herein may additionally or alternatively enable selection and programming of CSP pulse parameters for achieving conduction system capture and improved ventricular electrical synchrony with a high degree of confidence in a manner that is simplified, flexible, and patient-specific.

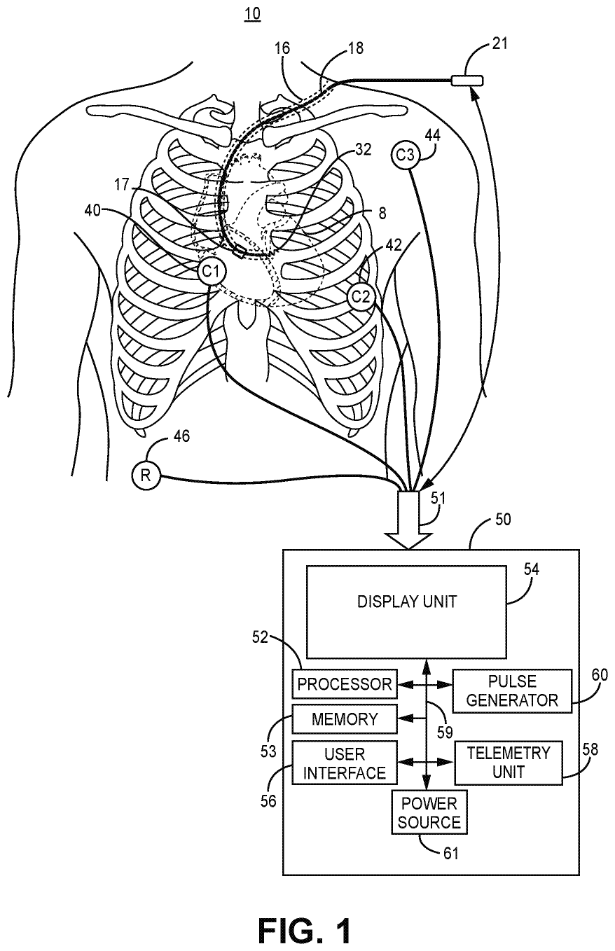

FIG. 1 is a conceptual diagram of a medical device system 10 capable of sensing and analyzing cardiac electrical signals and displaying a GUI including VATs determined by a processor receiving the cardiac electrical signals according to some examples. In FIG. 1, cardiac pacing lead 18 is shown advanced within a patient's heart 8 for positioning a pacing electrode 32 within the interventricular septum at a CSP site, e.g., at an LBB pacing site. It is to be understood, however, that a pacing electrode carried by a lead or by a housing of a leadless pacing device may be positioned at any desired CSP site. The system 10 includes an external device 50, which is also referred to herein as a “medical device,” for receiving and analyzing cardiac electrical signals during the pacing electrode implantation procedure and/or during follow-up monitoring or assessments. In the example shown, lead 18 may be coupled to external device 50 for delivering pacing pulses via pacing electrode 32, e.g., during an implant procedure. As described below, in other instances, lead 18 may be coupled to a pacemaker that is generating pacing pulses for delivery via pacing electrode 32 (and any return electrode) while external device 50 is analyzing ECG signals and generating data relating to CSP for display in a GUI External device 50 may be embodied as a programmer or pacing system analyzer used in a hospital, clinic or physician's office to acquire and analyze cardiac signals.

External device 50 may be a bedside or desktop device or a handheld device and may be a personal device such as a smartphone, tablet or other electronic device capable of receiving ECG signals, wirelessly or via connected ECG leads. In some examples, external device 50 is included in a remote patient monitoring system such as the CARELINK™ monitoring system available from Medtronic, Inc., Dublin, Ireland.

External device 50 may include an electrode/lead interface 51 for receiving input from ECG electrodes 40, 42, 44 and 46 and optionally from implantable pacing and sensing lead 18 via lead connector 21. External device 50 may include a processor 52, memory 53, display unit 54, user interface unit 56, telemetry unit 58, pulse generator 60 and power source 61. External device 50 may receive ECG signals sensed from a plurality of electrodes, which may be referred to as “chest” electrodes herein and can be cutaneous and/or subcutaneous electrodes in various examples. The term “chest” electrodes as used herein refers to electrodes that are placed in the thoracic region of the upper torso, e.g., below the neck and above the abdomen or below the sternum and above the lowest rib, and may be placed posteriorly, anteriorly or laterally on the patient.

In the example shown, three chest electrodes 40, 42 and 44 are positioned for receiving three input signals from which processing circuitry of external device 50 can determine up to three differential unipolar ECG signals and up to three bipolar ECG signals. The obtained ECG signals can be analyzed and/or displayed in a GUI with data derived from the obtained ECG signals for facilitating user ease of recognition of CSP capture and improved ventricular electrical synchrony during CSP. A reference electrode 46 may be positioned anywhere on the patient's body, e.g., along the right lower abdomen or any other location, which may be a chest, abdominal, limb or lower torso location, for serving as a common ground electrode for each of the raw input signals received from the C1, C2, and C3 electrodes 40, 42 and 44. In some examples, external device 50 includes an interface 51 for receiving four input signals, which include up to three chest electrode input signals and a reference electrode input signal. In general, the techniques disclosed herein provide improvements in displaying and presenting CSP related data that simplify the process of verifying CSP capture and improved ventricular electrical synchrony by reducing the total number of electrodes placed on the patient and the number of ECG signals received and analyzed by the medical device. The techniques disclosed herein provide improvements in displaying and presenting CSP related data that simplify the process of verifying CSP capture without requiring a 12-lead ECG system and a high level of electrophysiological expertise.

In the example shown, C1 electrode 40 is positioned in a right medial location, which may be between the second and fifth intercostal space to the right of the sternum. C1 electrode 40 may be positioned further to the left or right than the position shown and may be positioned posteriorly or anteriorly. As described below, an ECG signal obtained by processor 52 of external device 50 via C1 electrode 40 positioned as a right chest electrode may be representative of right ventricular electrical activity. The ECG signal obtained via C1 electrode 40 positioned as a right chest electrode may be displayed in a GUI for use in identifying a pathological RBB block or pacing-induced RBB block when the LBB is being paced. Cl electrode 40 may be positioned to sense a signal having a relatively greater contribution from the right ventricular depolarization than the left ventricular depolarization to enable identification of a RBB block type of QRS waveform. The C1 electrode 40 could be positioned to the left of the patient's sternum but may generally be positioned in a medial or rightward location, e.g., over the right ventricle or to the right of the right ventricle.

The C2 electrode 42 is shown positioned in a left lateral location and may be positioned between the third and seventh intercostal spaces (or third and eight ribs), as examples. The C2 electrode 42 may be positioned further to the left or right than the position shown in FIG. 1 but is generally positioned to the left of the C1 electrode 40. The C2 electrode 42 may be positioned anteriorly or posteriorly but is generally positioned to the left of the patient's sternum so that the ECG signal obtained via C2 electrode 42 is generally representative of electrical activity of the left ventricle. An ECG signal obtained via C2 electrode 42 may have a relatively greater contribution from the left ventricular depolarization than the right ventricular depolarization. As described below, a VAT can be determined from an ECG signal obtained via a left chest electrode, e.g., C2 electrode 42, and displayed on a GUI to facilitate user recognition of CSP capture in some examples.

The C3 electrode 44 is shown in an upper left lateral location in the example of FIG. 1. The C3 electrode 44 can be used to receive a third single-ended input signal. The third input signal can be used for determining a common mode signal or central terminal signal during processing of the input signals received from electrodes 40, 42, 44 and 46 and may be used in deriving one or more bipolar ECG signals in combination with unipolar ECG signals obtained from the input signals received from the C1 electrode 40 and/or C2 electrode 42. The position of C3 electrode 44 may vary between examples and may be any posterior or anterior position, in a right, left or medial location. The C1 electrode 40, C2 electrode 42 and C3 electrode 44 may be positioned to form a triangle that is approximately centered over the heart, e.g., which may be over the right ventricle, over the left ventricle, over the interventricular septum, over the ventricular apex or over the cardiac axis.

External device 50 may optionally receive a cardiac electrical signal via pacing lead 18. Pacing lead 18 may be electrically coupled to external device 50 for providing raw cardiac electrical signals received via pacing electrode 32 paired with another sensing electrode for obtaining a cardiac electrogram (EGM) signal, which may be displayed in a GUI with one or more ECG signals obtained from electrodes 40, 42, 44 and 46 and/or data derived therefrom.

Processor 52 may be coupled to the other components and units of external device 50, e.g., via a data bus 59, for controlling the functions attributed to external device 50 herein. For example, processor 52 may pass ECG and/or EGM signals and data derived therefrom to display unit 54 for displaying data in a GUI Processor 52 may control pulse generator 60, when included, to generate pacing pulses for delivery as CSP pulses. Processor 52 may control telemetry unit 58 to transmit and receive communication signals.

Processor 52 executes instructions stored in memory 53. Processor 52 may include any one or more of a microprocessor, a controller, a digital signal processor (DSP), an application specific integrated circuit (ASIC), a field-programmable gate array (FPGA), or equivalent discrete or analog logic circuitry. In some examples, processor 52 may include multiple components, such as any combination of one or more microprocessors, one or more controllers, one or more DSPs, one or more ASICs, or one or more FPGAs, as well as other discrete or integrated logic circuitry. The functions attributed to processor 52 herein may be embodied as software, firmware, hardware or any combination thereof.

Memory 53 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media. Memory 53 may be configured to store instructions executed by processor 52 for obtaining and analyzing ECG signals and generating data in a GUI according to the techniques disclosed herein. Memory 53 may store ECG signal features or parameters determined by processor 52 for use generating a display of CSP related data in a GUI as described below.

Display unit 54, which may include a liquid crystal display, light emitting diodes (LEDs) and/or other visual display components, may generate a display of the ECG and/or EGM signals and/or data derived therefrom. Display unit 54 may generate a GUI including various windows, icons, user selectable menus, etc. to facilitate interaction by a user with external device 50. Display unit 54 may function as an input and/or output device using technologies including liquid crystal displays (LCD), quantum dot display, dot matrix displays, light emitting diode (LED) displays, organic light-emitting diode (OLED) displays, cathode ray tube displays, e-ink, or monochrome, color, or any other type of display capable of generating tactile, audio, and/or visual output. In some examples, display unit 54 is a presence-sensitive display that may serve as a user interface device that operates both as one or more input devices and one or more output devices.

In some examples, display unit 54 may generate a display of at least one ECG signal annotated with VATs determined using an ECG signal sensed using a left chest electrode, e.g., C2 electrode 42. Processor 52 may be configured to determine the VATs for a plurality of paced cardiac cycles. As used herein, a “paced cardiac cycle” refers to a cardiac cycle during which a CSP pulse is delivered. It is to be understood, however, that the delivered CSP pulse may or may not capture any portion of the conduction system. The paced cardiac cycles may include cycles in which the delivered CSP pulse fails to capture any cardiac tissue (no pacing-evoked depolarization), captures only ventricular myocardium without capturing a portion of the conduction system, captures a combination of ventricular myocardium and at least a portion of the conduction system, and/or captures at least a portion of the conduction system without capturing ventricular myocardium.

In other examples, display unit 54 may produce an output to a user in another fashion, such as via a sound card, video graphics adapter card, speaker, one or more USB interfaces, video and/or audio output interfaces, or any other type of device capable of generating audio, video, or other output. For instance, display unit 54 may include a speaker configured to generate an audible notification in response to detecting a change in VAT by processor 52 that indicates a change from non-capture of the conduction system to capture of at least a portion of the conduction system or vice versa, for example.

User interface unit 56 may include a data entry or pointing device such as a mouse, touch screen, keypad or the like, to enable a user to interact with external device 50 and a GUI displayed on display unit 54, e.g., to initiate and terminate an implant session, adjust settings of display unit 54, enter programmable control parameters for programming into a pacemaker coupled to CSP lead 18, or make other user requests. Telemetry unit 58 includes a transceiver and antenna configured for bidirectional communication with a telemetry circuit included in an implantable pacemaker, which may be coupled to CSP lead 18 after pacing electrode 32 is deployed to an acceptable CSP site. Telemetry unit 58 is configured to operate in conjunction with processor 52 for sending and receiving data relating to pacemaker functions via a wireless communication link with the implantable pacemaker.

In some examples, external device 50 may include a pulse generator 60 for generating and delivering pacing pulses via lead 18 during the implant procedure. Post-pace ECG signals may be analyzed for determining when a VAT change is detected indicating improvement in electrical synchrony and an acceptable CSP site. In some examples, external device 50 may control pulse generator 60 to generate pacing pulses to perform capture tests during implantation of CSP lead 18. Lead 18 may be coupled to external device 50 via lead connector 21 and interface 51. Pulse generator 60 may include one or more holding capacitors charged to a pacing pulse voltage amplitude by a power source 61 of external device 50. The holding capacitor(s) may be coupled to an output capacitor via switching circuitry to deliver the pacing pulse via the pacing electrode 32 (and a return anode electrode) as the holding capacitor(s) are discharged for a selected pacing pulse width. In other examples, pulse generator 60 may be a separate device, such as a pacing system analyzer or temporary external pacemaker that can be coupled to lead 18 for delivering CSP pulses.

External device 50 includes a power source 61 that is coupled to the various units of external device 50 for providing power to circuits and components of external device 50 as needed. Power source 61 may include one or more rechargeable or non-rechargeable batteries or may be coupled to an external power source, such as plugged into an electrical outlet.

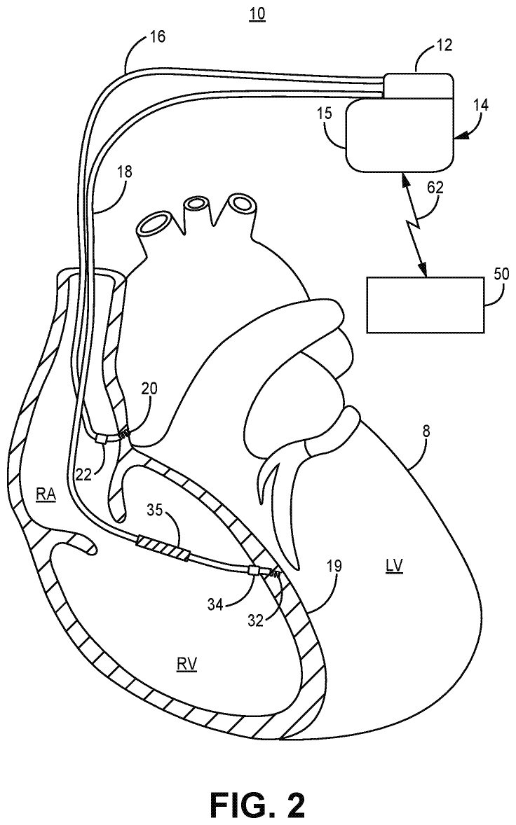

In some examples, a catheter 16 or other delivery tool used to implant lead 18 may include a return electrode 17 for use in combination with the pacing electrode 32 for delivering pacing pulses and/or sensing cardiac signals during implantation of pacing electrode 32 at a CSP site. Lead 18 may include one or more ring electrodes that may be selected for use in a pacing electrode vector, e.g., as shown in FIG. 2. However, in some instances a return electrode carried along lead 18 proximal from pacing electrode 32 may be insulated within the body of catheter 16 or another delivery tool such that it is not available for sensing and/or pacing during an implant procedure. In this case, catheter 16 may carry one or more electrodes, such as a ring return electrode 17, for use as an anode electrode paired with pacing electrode 32 prior to connecting lead connector 20 to an implantable pacemaker. In other examples, another cutaneous or subcutaneous electrode may be provided and coupled to external device 50 via interface 51 to serve as a return anode in combination with pacing electrode 32 for testing and assessment of CSP during an implant procedure.

FIG. 2 is a conceptual diagram of CSP lead 18 coupled to an implantable medical device (IMD) 14 capable of pacing a patient's heart 8 and sensing cardiac electrical signals via lead 18. Pacing electrode 32 of lead 18 can be positioned at an acceptable CSP site, which may be confirmed based at least in part on VAT data displayed in a GUI by external device 50 (as further described below). IMD 14 is shown as a dual chamber device configured to receive a right atrial lead 16, positioned in the right atrium (RA) for delivering atrial pacing pulses and sensing atrial electrical signals via electrodes 20 and 22. IMD 14 may be configured to sense intrinsic atrial P-waves and deliver atrial pacing pulses in the absence of sensed P-waves. IMD 14 may be configured to deliver atrial synchronized ventricular pacing by setting an AV delay in response to each sensed P-wave or delivered atrial pacing pulse and deliver a CSP pulse via lead 18 upon the expiration of the AV delay to pace the ventricles in synchrony with the atria.

CSP lead 18 may be advanced transvenously into the RV via the RA for positioning pacing electrode 32 within the inter-ventricular septum 19. Pacing electrode 32 can be referred to as a “tip electrode” because it is carried by CSP lead 18 at the distal lead tip. When pacing electrode 32 is advanced relatively superiorly within the inter-ventricular septum 19, pacing electrode 32 may be positioned along the inferior portion of the His bundle for delivering CSP pulses. In other examples, pacing electrode 32 may be advanced within the inter-ventricular septum 19 in the vicinity of a bundle branch of the His-Purkinje system, e.g., at a LBB pacing site in the area of the LBB or at a right bundle branch RBB pacing site in the area of the RBB, for delivering CSP pulses.

Pacing electrode 32 may be selected as a pacing cathode electrode in combination with ring electrode 34 as the return anode electrode for CSP. In some instances, the pacing pulse amplitude and pulse width may be selected to achieve cathodal capture at the cathode electrode for capturing at least at portion of one bundle branch. In other instances, the pacing pulse amplitude and pulse width may be selected to achieve cathodal and anodal capture, which may capture both the LBB and the RBB concurrently (by the same pacing pulse) to provide dual or bilateral bundle branch (BB) pacing using a single bipolar electrode pair. In other examples, either pacing electrode 32 or ring electrode 34 may be selected as the cathode electrode paired with housing 15 in a unipolar pacing electrode vector. Unipolar pacing may capture at least a portion of a single BB. In some cases, however, unipolar pacing may capture both the RBB and the LBB when a unipolar pacing pulse directly captures one bundle branch while virtual current or break excitation generated by the pacing electrode may excite the other bundle branch, potentially resulting in unipolar bilateral BB pacing, with capture of both the LBB and RBB.

While CSP lead 18 is shown carrying one pacing and sensing electrode pair, pacing electrode 32 and ring electrode 34, it is to be understood that in other examples, CSP lead 18 may include multiple pacing and sensing electrodes along its distal portion to provide one or more selectable bipolar pacing electrode vectors and/or one or more unipolar pacing electrode vectors (e.g., with housing 15) for delivering CSP pulses and sensing ventricular electrical signals.

CSP lead pacing 18 may further include one or more cardioversion/defibrillation (CV/DF) electrodes 35 for delivering relatively high voltage shock therapies. A CV/DF electrode generally has a high surface are and may be an elongated coil electrode as illustrated by coil electrode 35 on CSP lead 18. In addition to delivering relatively low voltage atrial and CSP pulses, IMD 14 can be configured as an implantable cardioverter defibrillator (ICD) capable of delivering high voltage shock therapies for terminating ventricular tachycardia or fibrillation. Coil electrode 35 may also be used in sensing electrode vectors, e.g., with either of pacing electrode 32 or ring electrode 34, for sensing a ventricular EGM signal that may be transmitted to external device 50 via communication link 62 for display in a GUI by display unit 54. Other examples of pacing lead configurations for delivering CSP that may be used in conjunction with the techniques described herein are generally disclosed in U.S. Publication No. 2022/0023640 (Zhou, et al.) and in U.S. patent Ser. No. 11/207,529 (Zhou), both of which are incorporated herein by reference in their entirety.

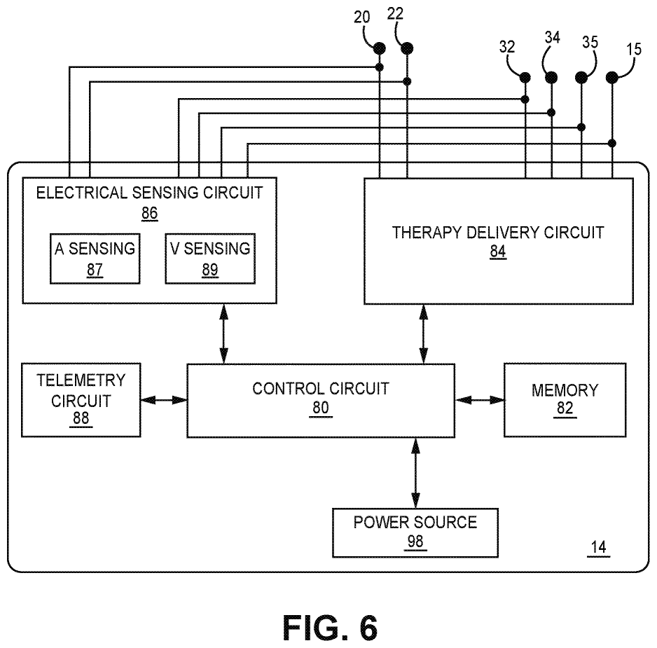

IMD 14 includes a housing 15 that encloses electronic circuitry configured to perform cardiac signal sensing and therapy delivery functions attributed to IMD 14. Examples of circuitry that may be included in IMD 14 are described below in conjunction with FIG. 6. IMD 14 includes a connector block 12, sometimes called a “header” having connector bores for receiving proximal lead connectors (not seen in FIG. 2) of each of the respective leads 16 and 18 coupled to IMD 14. Each of leads 16 and 18 include insulated electrical conductors extending through one or more lumens within the elongated, electrically insulating lead bodies of leads 16 and 18. Each electrical conductor extends from a respective electrode 20, 22, 32, 34 and 35 to the proximal lead connector of the corresponding lead 16 or 18 to provide electrical connection to electrical contacts within connector block 12. Electrical connection of the electrodes 20, 22, 32, 34 and 35 to internal electronic circuitry of IMD 14 is provided by electrical feedthroughs in connector block 12 that cross the hermetically sealed housing 15 of IMD 14.

In this way, the insulated electrical conductors extending through leads 16 and 18 carry electrical signals from therapy delivery circuitry within housing 15 to electrodes 20, 22, 32, 34 and 35 for delivering electrical stimulation therapies, performing impedance measurements, inducing tachyarrhythmia during device testing, etc. The insulated electrical conductors can carry cardiac electrical signals of heart 8 from electrodes 20, 22, 32, 34 and 35 to sensing circuitry within housing 15 for obtaining atrial and ventricular EGM signals. As described above, IMD 14 may communicate via wireless telemetry with external device 50. External device 50 may receive EGM signals, delivered atrial pacing pulse marker signals, and/or delivered CSP pulse marker signals that are transmitted by IMD 14 for use in analyzing ECG signals obtained by external device 50 and/or for display in a GUI for observation and review by a clinician or other user.

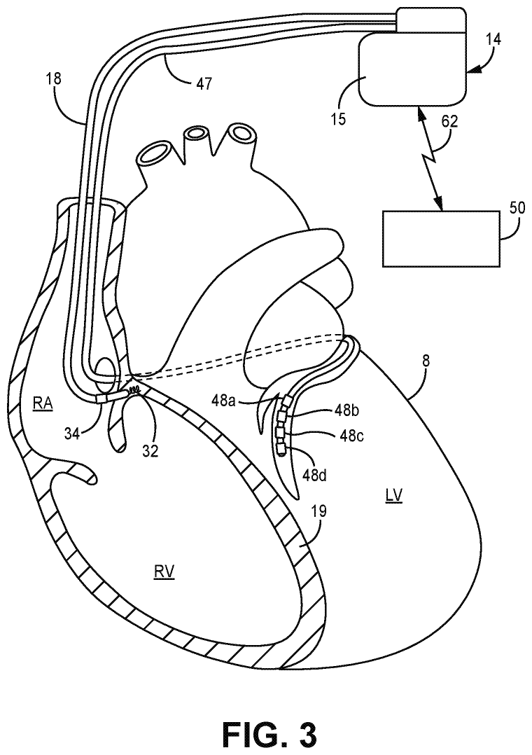

FIG. 3 is a conceptual diagram of IMD 14 coupled to CSP lead 18 advanced to an alternative location within the heart 8 for delivering CSP pulses and sensing cardiac electrical signals. In this example, the distal portion of CSP lead 18 is advanced within the RA for sensing ventricular electrical signals and delivering CSP pulses to or in the vicinity of the His bundle from a right atrial approach. In the example of FIG. 3, the pacing electrode 32 of CSP lead 18 can be advanced into the cardiac tissue in the area of the His bundle, e.g., between the His bundle and the coronary sinus and adjacent the tricuspid valve. A target entry site for pacing electrode 32 may correspond to or lie within the Triangle of Koch in some examples for achieving CSP at a His bundle pacing site. Pacing electrode 32 may be paired with the return anode ring electrode 34 for delivering CSP pulses and for sensing raw cardiac electrical signals, which may be processed for obtaining a ventricular EGM signal.

In some examples, CSP may be delivered in combination with LV myocardial pacing that can be delivered via a left ventricular (LV) lead 47 for further improvement in electrical and mechanical synchrony of the RV and LV, e.g., during cardiac resynchronization therapy (CRT). LV lead 47 may be advanced into the RA, through the coronary sinus ostium and into a cardiac vein of the left ventricle for positioning electrodes 48a, 48b, 48c and 48d (collectively “LV electrodes 48”) along the LV myocardium for sensing ventricular electrical signals and pacing the LV myocardium. LV lead 47 is shown as a quadripolar lead carrying four electrodes 48a-d that may be selected in various bipolar pacing electrode pairs for pacing the LV myocardial tissue and for sensing LV signals. One of LV electrodes 48 may be selected in combination with IMD housing 15 for delivering unipolar LV myocardial pacing in some instances and/or for sensing ventricular EGM signals that may be transmitted to external device 50 via communication link 62.

When CSP lead 18 is positioned for delivering CSP, CSP may be combined with ventricular myocardial pacing using LV lead 47 to correct an LV conduction delay and achieve electrical and mechanical synchrony of the ventricles. As such, in some examples, IMD 14 may control CSP pulse delivery in combination with LV myocardial pacing pulse delivery at specified time intervals which may include an AV delay and/or a ventricular-to-ventricular (VV) delay. The AV delay may control the timing of the CSP pulses and/or the LV myocardial pacing pulses relative to an atrial event, e.g., sensed P-wave or delivered atrial pacing pulse. In some examples, a VV delay may control the timing between a CSP pulse delivered via CSP lead 18 and an LV myocardial pacing pulse delivered via LV lead 47.

It is to be understood that LV lead 47 is optional. In some examples, IMD 14 is coupled only to CSP lead 18 advanced into the RA or the RV for positioning pacing electrode 32 at a CSP site for delivering CSP and sensing ventricular EGM signals. In other examples, RA lead 16 as shown in FIG. 2 is implanted in combination with the CSP lead 18 for delivering CSP in a dual chamber sensing and pacing system. External device 50 may receive one or more EGM signals from IMD 14 sensed using any available EGM sensing electrode vector.

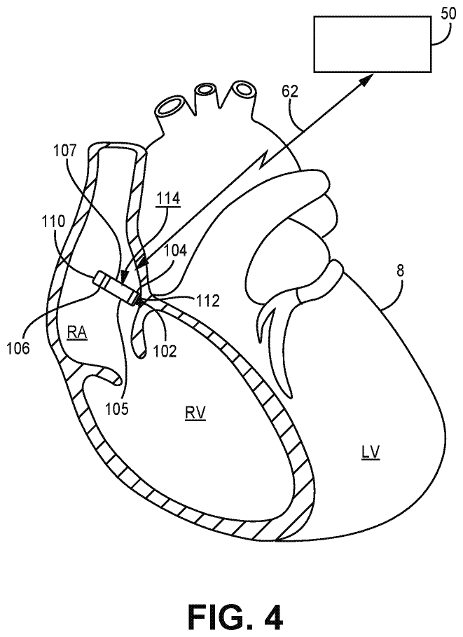

FIG. 4 is a conceptual diagram of a leadless pacemaker 114 positioned within the RA for providing CSP according to another example. Pacemaker 114 may include a distal tip electrode 102, which can also be referred to as a “pacing electrode” or “CSP electrode” extending away from a distal end 112 of the pacemaker housing 105. Pacemaker 114 is shown implanted in the RA of the patient's heart 8 to place distal tip electrode 102 for delivering CSP pulses in the area of the His bundle. For example, the distal tip electrode 102 may be inserted into the inferior end of the interatrial septum, beneath the AV node and near the tricuspid valve annulus to position tip electrode 102 in, along or proximate to the His bundle. Distal tip electrode 102 may be a helical electrode providing fixation to anchor the pacemaker 114 at the implant position. In other examples, pacemaker 114 may include a fixation member that includes one or more tines, hooks, barbs, helices or other fixation member(s) that anchor the distal end of the pacemaker 114 at the implant site. A portion of the distal tip electrode 102 may be electrically insulated such that only the most distal end of tip electrode 102, furthest from housing distal end 112, is exposed to provide targeted pacing at a CSP site.

One or more housing-based electrodes 104 and 106 may be carried on the surface of the housing 105 of pacemaker 114. Electrodes 104 and 106 are shown as ring electrodes circumscribing the longitudinal sidewall 107 of pacemaker housing 105. Longitudinal sidewall 107 extends from distal end 112 to proximal end 110 of housing 105. In other examples, a return anode electrode used in sensing and pacing may be positioned on housing proximal end 110. Pacing of the conduction system may be achieved using the distal tip electrode 102 as the cathode electrode and either of the housing-based electrodes 104 and 106 as the return anode.

Cardiac electrical signals may be sensed by pacemaker 114 using a sensing electrode pair selected from electrodes 102, 104 and 106. For example, a cardiac electrical signal may be sensed using distal tip electrode 102 and distal housing-based electrode 104 or proximal housing-based electrode 106. A second cardiac electrical signal may be sensed using electrodes 104 and 106. An EGM signal sensed by pacemaker 114 may be transmitted to external device 50 via communication link 62 for display in a GUI.

In some examples, atrial P-waves may be sensed from a signal received via electrodes 104 and 106 and/or atrial pacing pulses may be delivered via electrodes 104 and 106. Atrial synchronous CSP pulses may be delivered via electrodes 102 and 104 at an AV delay following sensed atrial P-waves and/or delivered atrial pacing pulses.

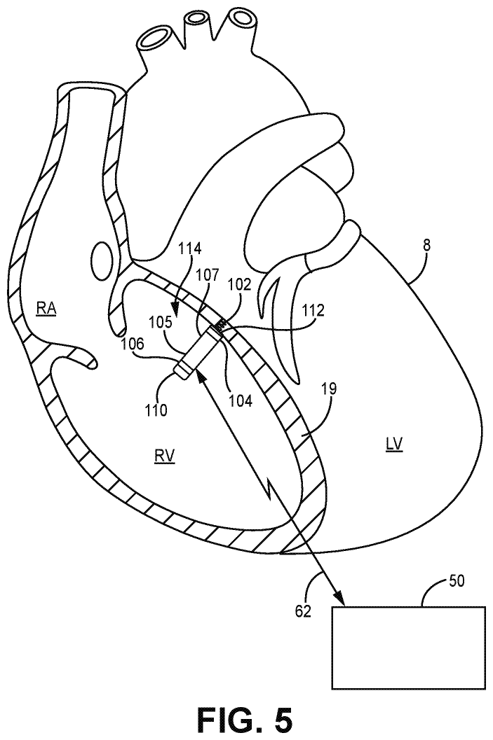

FIG. 5 is a conceptual diagram of the leadless pacemaker 114 of FIG. 4 shown implanted in an alternative location for CSP. Pacemaker 114 may be implanted within the RV along the inter-ventricular septum 19 for providing CSP in some examples. Techniques disclosed herein may be used in conjunction with a leadless pacemaker, such as pacemaker 114, having a pacing electrode 102 coupled to and extending directly from the pacemaker housing 105, without requiring an intervening medical lead coupled to the pacemaker 114 for carrying the pacing and sensing electrode(s).

In this example, pacemaker 114 may be positioned within the RV for advancing the pacing tip electrode 102 extending from the distal end 112 of pacemaker housing 105 into the inter-ventricular septum 19 for delivering CSP, e.g., in the area of an inferior portion of the His bundle or along one or both of the RBB and LBB depending on the relative positioning of distal tip electrode 102. Distal tip electrode 102 is shown as a “screw-in” helical electrode but may be configured as other types of tissue-piercing electrodes capable of being advanced within the septal tissue. A proximal portion of the distal tip electrode 102 may be electrically insulated, e.g., with a coating, such that only a distal portion of tip electrode 102, furthest from pacemaker housing distal end 112, is exposed to provide targeted pacing at a tissue site that includes the His bundle, LBB and/or RBB.

In other examples, distal tip electrode 102 may be formed having a straight shaft with a distal active electrode portion or other type of electrode, which may be a tissue-piercing electrode that is advanceable through the inter-ventricular septum 19 to deliver CSP, e.g., in a left portion of the septum 19 in the area of the LBB. In some examples, pacemaker 114 may include a fixation member that includes one or more tines, hooks, barbs, helices or other fixation member(s) that anchor the distal end 112 of the pacemaker 114 at the implant site and may not function as an electrode. Examples of leadless intracardiac pacemakers that may be configured for delivering cardiac pacing pulses to the conduction system that may be used in conjunction with the techniques described herein are generally disclosed in the above-incorporated U.S. Pat. No. 11,207,529 (Zhou) and in U.S. Publication No. 2019/0083800 (Yang, et al.), incorporated herein by reference in its entirety.

Pacemaker 114 may include the distal housing-based ring electrode 104 along or near the distal end 112 of pacemaker housing 105. In an example, distal housing-based ring electrode 104 may be selectable as the return anode electrode with distal tip electrode 102 for bipolar pacing of the LBB and/or RBB in the vicinity of the distal tip electrode 102. Bipolar bilateral BB pacing of both the RBB and LBB simultaneously may be achieved by cathodal capture of the LBB at distal tip electrode 102 and anodal capture of the RBB by distal ring electrode 104. The polarities of the distal tip electrode 102 and the distal ring electrode 104 may be reversed to achieve cathodal capture of the RBB and anodal capture of the LBB in some examples. Distal ring electrode 104 is shown as a ring electrode circumscribing a distal portion of the housing 105 but may alternatively be a distal housing-based electrode in the form of a button electrode, hemispherical electrode, segmented electrode or the like and may be along the face of distal end 112 of housing 105 and/or along longitudinal sidewall 107.

In the example shown, a housing-based proximal ring electrode 106, which may circumscribe all or a portion of the longitudinal sidewall 107 of the housing 105, may be provided as a return anode electrode. In other examples, a return anode electrode used in sensing and pacing may be positioned on housing proximal end 110 and may be a button, ring or other type of electrode. CSP in the area of the LBB may be achieved using the tip electrode 102 as the cathode electrode and the proximal ring electrode 106 as the return anode. CSP in the area of the RBB and/or myocardial tissue of inter-ventricular septum 19 may be achieved using the distal ring electrode 104 as a cathode electrode and the proximal ring electrode 106 as the return anode. In this way, bilateral or dual bundle branch pacing of the conduction system may be achieved using two different bipolar pacing electrode vectors carried by housing 105.

Cardiac electrical signals produced by heart 8 may be sensed by pacemaker 114 using electrodes 102, 104 and/or 106. The cardiac electrical signal received via electrodes 102 and 104, electrodes 104 and 106 and/or electrodes 102 and 106, for example, may be sensed by pacemaker 114 and processed by processing circuitry of IMD 14 and/or transmitted wirelessly, e.g., as EGM signals, to external device 50 via communication link 62. The EGM signals may then be displayed and/or further processed and analyzed by the processor 52 of external device 50 for providing a user with visual representations of sensed EGM signals and/or CSP related data.

The examples of FIGS. 1-5 present various lead and/or electrode configurations that may be implemented for delivering CSP in a medical device system configured to perform the techniques disclosed herein for analyzing cardiac electrical signals and generating a GUI for presenting data to a clinician or other user for confirming a CSP site of a pacing electrode and/or confirming CSP capture. The various lead and electrode configurations described and shown in the accompanying drawings are intended to be illustrative in nature. It is to be understood that the leads and electrodes illustrated in FIGS. 1-5 may be implanted in different combinations and/or other locations than the examples shown and some leads and/or electrodes may be omitted or additional leads and/or electrodes may be provided in a medical device system configured to deliver and monitor CSP. In some examples, a leadless IMD, e.g., pacemaker 114, may be implanted in a patient for CSP in combination with another implanted IMD, e.g., an IMD connected to a RA lead for pacing and sensing in the right atrium and/or an ICD coupled to extracardiac leads for providing tachyarrhythmia detection and therapy delivery. A variety of lead-based and leadless IMD configurations may be conceived for sensing cardiac electrical signals and delivering CSP pulses which may be used in conjunction with the techniques disclosed herein for analyzing ECG signals and presenting CSP related data to a user in GUI displayed by external device 50.

FIG. 6 is a schematic diagram of circuitry that may be enclosed within an IMD configured to sense cardiac electrical signals and perform CSP. The block diagram of FIG. 6 is described with reference to IMD 14 coupled to electrodes 20 and 22 carried by RA lead 16 and electrodes 32, 34 and 35 carried by CSP lead 18 as shown in FIG. 2, as an illustrative example. It is to be understood, however, that the functionality attributed to the various circuits and components shown in FIG. 6 for sensing cardiac signals and delivering CSP may be implemented in conjunction with other lead and electrode configurations, including the leadless pacemaker 114 of FIGS. 4 and 5 or other medical devices configured to deliver CSP pulses and sense cardiac electrical signals.

Housing 15 is represented as an electrode in FIG. 6 for use in cardiac electrical signal sensing and, in some examples, for delivery of unipolar pacing pulses. When IMD 14 is implemented as an ICD, housing 15 may be used as an active can electrode for delivery of CV/DF shock pulses. The electronic circuitry enclosed within housing 15 includes software, firmware and hardware that cooperatively monitor cardiac electrical signals, determine when a pacing pulse is necessary, and deliver electrical pacing pulses to the patient's heart as needed according to a programmed pacing mode and pacing pulse control parameters. The electronic circuitry can include a control circuit 80, memory 82, therapy delivery circuit 84, cardiac electrical signal sensing circuit 86, telemetry circuit 88, and power source 98.

Power source 98 provides power to the circuitry of IMD 14 including each of the components 80, 82, 84, 86, and 88 as needed. Power source 98 may include one or more energy storage devices, such as one or more rechargeable or non-rechargeable batteries. The connections between power source 98 and each of the other components 80, 82, 84, 86, and 88 are to be understood from the general block diagram of FIG. 6 but are not shown for the sake of clarity. For example, power source 98 may be coupled to one or more charging circuits included in therapy delivery circuit 84 for providing the power needed to charge holding capacitors included in therapy delivery circuit 84 that are discharged at appropriate times under the control of control circuit 80 for delivering pacing pulses. Power source 98 is also coupled to components of sensing circuit 86, such as sense amplifiers, analog-to-digital converters, switching circuitry, etc. as needed for sensing cardiac electrical signals. Power source 98 may provide power to the various components and circuits of telemetry circuit 88 and memory 82 as needed, which may be under the control of control circuit 80.

The circuits shown in FIG. 6 represent functionality included in IMD 14 and may include any discrete and/or integrated electronic circuit components that implement analog and/or digital circuits capable of producing the functions attributed to IMD 14 (or pacemaker 114) herein. The various components may include an application specific integrated circuit (ASIC), an electronic circuit, a processor (shared, dedicated, or group) and memory that execute one or more software or firmware programs, a combinational logic circuit, state machine, or other suitable components or combinations of components that provide the described functionality. Providing software, hardware, and/or firmware to accomplish the described functionality in the context of any modern cardiac medical device system, given the disclosure herein, is within the abilities of one of skill in the art.

Control circuit 80 communicates, e.g., via a data bus, with therapy delivery circuit 84 and sensing circuit 86 for cooperatively sensing cardiac electrical signals and controlling delivery of cardiac electrical stimulation therapies in response to sensed cardiac event signals, e.g., P-waves attendant to atrial depolarizations and R-waves attendant to ventricular depolarizations, or the absence thereof. The available electrodes are electrically coupled to therapy delivery circuit 84 for delivering electrical stimulation pulses and/or to sensing circuit 86 for sensing cardiac electrical signals produced by the heart. Sensed cardiac electrical signals may include both intrinsic signals (such as intrinsic P-waves and intrinsic R-waves) produced by the heart in the absence of a pacing pulse that captures the heart and evoked response signals following a delivered pacing pulse of sufficient energy to cause capture of cardiac tissue.

Sensing circuit 86 may include one or more sensing channels for receiving raw cardiac electrical signals from one or more sensing electrode vectors. For example, an atrial signal may be sensed using right atrial lead electrodes 20 and 22 coupled to atrial sensing (A sensing) channel 87. A ventricular signal may be sensed by ventricular sensing (V sensing) channel 89 using electrodes 32, 34 and/or 35 carried by CSP lead 18. In some examples, V sensing channel 89 may include multiple ventricular sensing channels for receiving raw signals from multiple sensing electrode vectors that may include at least one electrode in or proximate to the ventricular chambers. For instance, V sensing channel 89 may include a near field sensing channel for receiving a raw near field signal using electrodes 32 and 34 of CSP lead pacing 18 in a bipolar sensing electrode pair. V sensing channel 89 may include a far field or unipolar sensing channel for receiving a raw far field signal. For example, a raw far field signal may be received using a second electrode vector having electrodes spaced further apart than the electrodes of the near field sensing electrode vector. A far field signal may be sensed, for example, using pacing electrode 32 or ring electrode 34 of CSP lead 18 paired with IMD housing 15. In some examples, V sensing channel 89 may receive a raw far field signal sensed using pacing electrode 32 or ring electrode 34 paired with coil electrode 35. In other examples, a far field signal may be sensed using coil electrode 35 paired with IMD housing 15.

Sensing circuit 86 may include switching circuitry for selectively coupling a sensing electrode pair from the available electrodes to a respective sensing channel of A sensing channel 87 or V sensing channel 89. Switching circuitry may include a switch array, switch matrix, multiplexer, or any other type of switching device suitable to selectively couple components of sensing circuit 86 to selected electrodes.

Each of the sensing channels 87 and 89 of sensing circuit 86 may include an input filter for receiving a raw cardiac electrical signal from a respective pair of sensing electrodes, a pre-amplifier, an analog-to-digital converter (ADC), and a bandpass filter for producing a multi-bit digital cardiac electrical signal, which may be referred to as an “intracardiac EGM” signal when the raw signal is sensed using at least one electrode within a heart chamber. A multi-bit EGM signal may be passed from sensing circuit 86 to control circuit 80 for processing and analysis and/or for transmission to external device 50 (e.g., shown in FIG. 1) for processing and analysis and/or display on display unit 54.

Each sensing channel 87 and 89 may include cardiac event detection circuitry, which may include one or more sense amplifiers, filters, rectifiers, threshold detectors, comparators, analog-to-digital converters (ADCs), timers or other analog or digital components, for detecting cardiac electrical event signals. For example, an atrial event detector may be included in A sensing channel 87 for sensing intrinsic P-waves attendant to intrinsic atrial depolarizations using one or both of electrodes 20 and 22 carried by right atrial lead 16. A ventricular event detector may be included in V sensing channel 89 for sensing intrinsic R-waves attendant to intrinsic ventricular depolarizations using electrodes 32 and 34 carried by CSP lead 18.

A cardiac event sensing threshold, such as a P-wave sensing threshold and/or an R-wave sensing threshold, may be automatically adjusted by sensing circuit 86 under the control of control circuit 80, e.g., based on timing intervals and sensing threshold values determined by control circuit 80, stored in memory 82, and/or controlled by hardware, firmware and/or software of control circuit 80 and/or sensing circuit 86. The R-wave sensing threshold, for example, may be controlled to start at a starting threshold voltage following a post-ventricular blanking period then decrease according to a decay profile until reaching a minimum sensing threshold. The minimum R-wave sensing threshold may be set to a programmed sensitivity of the R-wave detection circuitry. The sensitivity, programmed to a voltage level, typically in millivolts, is the lowest voltage level above which a cardiac event, e.g., a P-wave or an R-wave, can be sensed by the cardiac event detection circuitry of the respective A sensing channel 87 or V sensing channel 89.

Upon detecting a cardiac electrical event signal based on a sensing threshold crossing, sensing circuit 86 may produce a sensed event signal that is passed to control circuit 80. For example, an atrial event detector may produce an atrial sensed event signal in response to a P-wave sensing threshold crossing. A ventricular event detector may produce a ventricular sensed event signal in response to an R-wave sensing threshold crossing. The sensed event signals can be used by control circuit 80 for starting pacing escape interval timers that control the basic time intervals used for scheduling cardiac pacing pulses, e.g., atrial pacing pulses and CSP pulses.

Control circuit 80 may include various timers or counters for counting down an AV delay, a VV delay, an atrial pacing lower rate interval, a ventricular pacing lower rate interval, or other pacing escape intervals according to a pacing mode and pacing control parameters. A sensed event signal may trigger or inhibit a pacing pulse depending on the particular programmed pacing mode. For example, a P-wave sensed event signal received from sensing circuit 86 may cause control circuit 80 to inhibit a scheduled atrial pacing pulse and schedule a CSP pulse at an AV delay. If the AV delay expires before control circuit 80 receives an R-wave sensed event signal from sensing circuit 86, therapy delivery circuit 84 may generate and deliver a CSP pulse at the AV delay following the sensed P-wave and in this way deliver atrial-synchronized ventricular pacing. If an R-wave sensed event signal is received from sensing circuit 86 before the AV delay expires, the scheduled CSP pulse may be inhibited. The AV delay controls the amount of time between an atrial event, paced or sensed, and a CSP pulse to promote electrical and mechanical synchrony of the heart chambers.

In other instances, a ventricular pacing lower rate interval (LRI) may be set by control circuit 80 to schedule a CSP pulse following a delivered CSP pulse or sensed R-wave. The LRI may correspond to a programmed ventricular lower rate or may be adjusted to a temporary LRI by control circuit 80 to deliver rate response pacing when an increase in patient activity level is detected, e.g., by an accelerometer signal or other patient activity sensor included in IMD 14 (not shown in FIG. 6). When IMD 14 is operating in a dual chamber pacing mode, e.g., a DDD mode, when a P-wave is sensed during the LRI, a CSP pulse can be triggered to occur at the AV delay, and the LRI can restarted upon delivery of the CSP pulse. If the LRI expires without a sensed P-wave or a sensed R-wave, the CSP pulse can be delivered at the expiration of the LRI, and the LRI can be restarted. Control circuit 80 may be configured to control therapy delivery circuit 84 to deliver CSP pulses according to a variety of pacing modes and pacing therapies, which may include bradycardia pacing, post-shock pacing, anti-tachycardia pacing (ATP), cardiac resynchronization therapy (CRT), rate response pacing, etc.

Therapy delivery circuit 84 may include charging circuitry, one or more charge storage devices such as one or more holding capacitors, an output capacitor, and switching circuitry that controls when the holding capacitor(s) are charged and discharged across the output capacitor to deliver a pacing pulse to a selected pacing electrode vector coupled to the therapy delivery circuit 84. Therapy delivery circuit 84 may include one or more pacing channels. In the example of IMD 14, therapy delivery circuit 84 may include an atrial pacing channel and a ventricular pacing channel each including one or more holding capacitors, one or more switches, and an output capacitor for producing pacing pulses delivered by the respective RA lead 16 (e.g., via electrodes 20 and 22) or CSP lead 18 (e.g., via electrodes 32 and 34). In other examples, the atrial and ventricular pacing pulses may be generated and delivered by shared pulse generating circuitry.

Charging of a holding capacitor to a programmed pacing voltage amplitude and discharging of the capacitor for a programmed pacing pulse width may be performed by therapy delivery circuit 84 according to control signals received from control circuit 80. For example, a pace timing circuit included in control circuit 80 may include programmable digital counters set by a microprocessor of the control circuit 80 for controlling the basic pacing time intervals associated with various single chamber and/or dual chamber pacing modes, multi-chamber pacing modes when LV lead 47 (shown in FIG. 3) is connected to IMD 14 for delivering CRT, and/or for delivering ATP sequences, as examples. The microprocessor of control circuit 80 may also set the amplitude, pulse width, polarity or other characteristics of the cardiac pacing pulses, which may be based on programmed values stored in memory 82.

IMD 14 may be configured to detect non-sinus tachycardia and deliver ATP. When IMD 14 is configured as an ICD for detecting tachyarrhythmia and delivering CV/DF shocks, therapy delivery circuit 84 may include high voltage therapy delivery circuitry for generating high voltage shock pulses in addition to low voltage therapy circuitry for generating low voltage pacing pulses. In response to detecting ventricular tachycardia or fibrillation, control circuit 80 may control therapy delivery circuit 84 to deliver a CV/DF shock. The high voltage therapy circuitry may include high voltage capacitors and high voltage charging circuitry for generating and delivering CV/DF shock pulses using elongated coil electrodes, e.g., coil electrode 35, carried by one or more leads coupled to IMD 14 and/or housing 15.

Control parameters utilized by control circuit 80 for sensing cardiac event signals (e.g., P-waves and R-waves) and controlling pacing therapy delivery may be programmed into memory 82 via telemetry circuit 88. Telemetry circuit 88 includes a transceiver and antenna for communicating with external device 50 (e.g., shown in FIG. 1) using radio frequency communication or other communication protocols as described above. Under the control of control circuit 80, telemetry circuit 88 may receive downlink telemetry from and send uplink telemetry to the external device 50. In some cases, telemetry circuit 88 may be used to transmit and receive communication signals to/from another medical device implanted in the patient. Telemetry circuit 88 can transmit EGM signals, pacing pulse timing markers, atrial and ventricular sensed event signal markers and other sensing and pacing related data for receipt by external device 50 in real time and/or from stored EGM signal episodes, which may be displayed by external device 50.

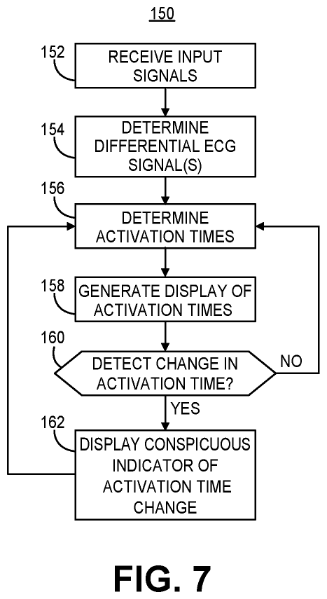

FIG. 7 is a flow chart 150 of a method for processing and analyzing ECG signals and generating a GUI for display by external device 50 according to some examples. With reference to external device 50 shown in FIG. 1, external device processor 52 receives input signals from multiple electrodes positioned on the patient at block 152. External device processor 52 may be configured to receive an input signal from each of four or less electrodes, e.g., consisting of three or less chest electrodes and a reference electrode. External device processor 52 may be configured to receive and analyze input signals consisting of five or less input signals received from each of five or less electrodes consisting of four or less chest electrodes and a reference electrode in other examples. In other examples, external device processor 52 may be configured to receive an input signal from at least two chest electrodes, e.g., a right chest electrode and a left chest electrode, and a reference electrode. In still other examples, external device processor 52 may be configured to receive input signals from a plurality of chest electrodes comprising two, three, four or more chest electrodes and a reference electrode where the plurality of chest electrodes includes at least one right chest electrode and at least one left chest electrode and may consist of four or less electrodes, five or less electrodes, six or less electrodes, seven or less electrodes, eight or less electrodes, or nine or less electrodes. A right chest electrode may be positioned medially, e.g., over the sternum or spine, or to the right of the sternum or spine for sensing a cardiac electrical signal representative of or correlated to right ventricular electrical activity. A left chest electrode may be positioned over the sternum or spine but may generally be positioned to the left of the sternum or spine to sense a cardiac electrical signal representative of or correlated to left ventricular electrical activity. Each input signal may be received as a single-ended input signal.

A 12-lead ECG can be manually analyzed by an electrophysiologist or other expert for observing and recognizing conduction system abnormalities and improvements in the conduction system abnormalities. A 12-lead ECG generally requires 10 electrodes including limb electrodes and precordial electrodes that can be time consuming to position for obtaining high quality ECG signals. Observation and analysis of a 12-lead ECG requires considerable expertise and time for identifying capture of the conduction system and improvement in ventricular electrical synchrony. Some clinics or other medical facilities that may be treating or following up a patient implanted with an IMD or pacemaker for delivering CSP may not be equipped or staffed for performing 12-lead ECG studies for monitoring CSP and making any necessary adjustments to promote CSP capture and improved ventricular electrical synchrony. The techniques disclosed herein enable a user to connect fewer electrodes to external device 50 than the ten electrodes required for 12 lead ECG studies and present CSP related data determined from obtained ECG signals in a GUI that simplifies for a user the process of verifying CSP capture and ventricular electrical synchrony improvement in a time efficient manner.

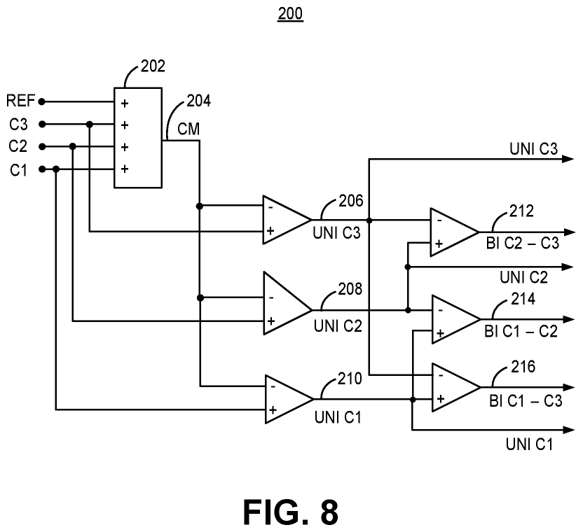

At block 154, external device processor 52, referred to hereafter as “processor” 52, may obtain one or more ECG signals from the received input signals by determining one or more differential ECG signals from the input signals. As described below in conjunction with FIG. 8, processor 52 may determine a common mode voltage signal that may be representative of a central terminal of the chest electrodes. Processor 52 may determine a unipolar left chest ECG signal using the input signal received via a left chest electrode and the common mode voltage signal, also referred to herein as a “central terminal signal.” Processor 52 may determine a unipolar right chest ECG signal using the input signal received via a right chest electrode and the common mode voltage signal Processor 52 may determine a bipolar ECG signal by determining the difference between two unipolar ECG signals. In some examples, processor 52 may determine a plurality of bipolar ECG signals, where each bipolar ECG signal can be computed as the difference between two different unipolar ECG signals.

At block 156, processor 52 determines a VAT from at least one of the differential ECG signals. Processor 52 may determine a VAT using a unipolar left chest ECG signal. In one example, processor 52 determines the VAT from the unipolar left chest ECG signal by determining the time interval from a delivered CSP pulse to a maximum peak amplitude of the unipolar left chest ECG signal, which may be a rectified or non-rectified signal. In another example, processor 52 determines the VAT using a unipolar left chest ECG signal by computing a bipolar ECG signal using the unipolar left chest ECG signal and a second unipolar ECG signal determined from the input signals. The VAT may be determined by processor 52 by determining the time interval from a delivered CSP pulse to a maximum peak amplitude of a bipolar ECG signal computed using the input signal received from the left chest electrode. The left chest electrode may be located between the sternum and the spine on the left side of the patient between a third intercostal space and the eighth intercostal space, as examples.

VATs (referred to as “activation times” in flow chart 150 and other drawings presented herein) may be determined by processor 52 from at least one ECG signal, unipolar or bipolar, at block 156 using an input signal received from a left chest electrode. A VAT may be determined for each of a plurality of cardiac cycles, e.g., from each of a plurality of delivered CSP pulses to a respective maximum peak amplitude of the ECG signal. VATs may be determined beat-by-beat (e.g., pulse by pulse in a series of multiple CSP pulses) or for non-consecutive cardiac cycles. At block 158, processor 52 in cooperation with display unit 54 of external device 50 generates a display of the determined VATs in a GUI.

As described below and shown in the accompanying drawings, processor 52 in cooperation with display unit 54 may generate a display of at least one ECG signal determined from the input signals that may be annotated or shown in combination with the determined VATs on a beat-by-beat basis. In some examples, the beat-by-beat display of at least one ECG signal and associated VATs may be displayed as a scrolling ECG signal in real time or as a previously stored ECG episode after processing and analysis and data collection. The scrolling ECG signal and associated VATs may be frozen on display unit 54 by a user interacting with the GUI, e.g., via a touch screen or other pointing device of user interface 56 for inspection by the user.

In other examples, at least one ECG signal and associated VATs may be displayed in the GUI on a beat-by-beat basis as individual cardiac cycles, e.g., one paced cardiac cycle, at a time. The single cardiac cycle displayed individually may be updated on a continuous beat-by-by-beat basis. One pacing pulse marker followed by a QRS waveform and an associated VAT may be displayed in a window of the GUI on display unit 54 and may be updated beat-by-beat. The single cycle beat-by-beat display may be frozen by a user to enable closer inspection. The display of a scrolling signal of multiple cardiac cycles with associated VATs and the display of an individual cardiac cycle updated beat-by-beat may be toggled between by a user interacting with the GUI, e.g., via a touch screen or other pointing device of user interface 56.

In some examples, VATs may be determined at block 156 from a delivered CSP pulse to a maximum peak amplitude (or other selected fiducial point of the post-pace QRS waveform) of one or more ECG signals, unipolar or bipolar, in addition to the VAT determined from a unipolar (or bipolar) left chest ECG signal. For example, a VAT may be determined from a CSP pulse to a maximum peak amplitude of an ECG signal, unipolar or bipolar, determined using in an input signal from a right chest electrode. Two or more ECG signals may be displayed in the GUI in combination with the VATs determined from the ECG signals sensed during CSP and displayed for each of a plurality of cardiac cycles. When VATs are determined from multiple ECG signals, each ECG signal may be annotated with the associated VATs determined from that respective ECG signal.

Each of the VATs displayed at block 158 may be formatted based on a value of the respective VAT. For example, when the VAT is greater than or equal to a threshold value, e.g., greater than 100 milliseconds (ms) or another threshold value, the VAT may be displayed in a red font or otherwise formatted to indicate that the VAT is unlikely to be associated with capture of the conduction system. Loss of capture or capture of ventricular myocardium only without conduction system capture may be occurring when the VAT is greater than the threshold value. When the VAT is less than the threshold value, indicating at least a portion of the conduction system is likely being captured, the VAT may be displayed in a green font or otherwise formatted to indicate likely capture of the conduction system.

At block 160, processor 52 may compare a VAT determined for a current cardiac cycle to a VAT determined for a previous cardiac cycle. In some examples, processor 52 may determine a VAT difference between VATs determined from two different cardiac cycles, which may or may not be consecutive cardiac cycles. Processor 52 may detect a change in VAT at block 160 based on the difference (or the ratio or other comparative analysis) between a current or most recent VAT and a previous VAT. The change in VAT may be detected at block 160 when the difference (or other quantitative relationship) between two VATs is equal to or greater than a threshold difference. The threshold difference may be 10 ms, 20 ms, 30 ms, 40 ms, 50 ms, 60 ms or any other threshold difference. The VAT difference may be determined between two consecutively determined VATs from two consecutive CSP cycles. In other examples, the VAT difference meeting the difference threshold may be two VATs determined within three consecutive CSP cycles, five CSP cycles, eight CSP cycles or other threshold number of CSP cycles.

Processor 52 may detect a sudden change in VAT when the difference threshold is met by two consecutively determined VATs in some examples, which may or may not be determined from consecutively occurring cardiac cycles but may be associated with two different CSP pulses delivered at different pacing sites, by different pacing electrode vectors and/or having different pacing pulse output (different pulse amplitude and/or pulse width). A change in VAT from a first VAT to a second VAT that are different by at least a difference threshold can occur when a change in the delivery of CSP pulses results in a change from no capture of the conduction system to capture of the conduction system or vice versa. Thus, processor 52 may detect a change in VAT from one paced cardiac cycle to another paced cardiac cycle when the pacing site, pacing electrode vector, and/or pacing pulse output changes from one paced cardiac cycle to another paced cardiac cycle.

In some examples, a VAT may be a representative VAT determined from multiple VATs determined during paced cardiac cycles when the CSP pulses are delivered in the same way (same pacing site, pacing electrode vector and pacing pulse output). Processor 52 may determine a first representative VAT as a mean, median, maximum, minimum or other representative value from a first plurality of VATs (e.g., two, three, four, six, eight or other selected number of VATs) determined from paced cardiac cycles when the CSP pulses are delivered using a first pacing pulse output (e.g., first pulse amplitude and first pulse width) and a first pacing electrode vector at a first pacing site. A second representative VAT may be determined as a mean, median, maximum, minimum or other representative VAT determined from a second plurality of VATs determined from paced cardiac cycles when the CSP pulses are delivered using a second pacing pulse output and/or second pacing electrode vector and/or second pacing site, wherein the second pacing pulse output, second pacing electrode vector, and second pacing site can each be different than the first pacing pulse output, first pacing electrode vector, and first pacing site, respectively. Processor 52 may detect the change in VAT at block 160 based on a threshold difference being met by the first representative VAT and the second representative VAT.

It is to be understood that in some instances the consecutive CSP cycles may not be consecutive cardiac cycles in that one CSP cycle may be separated from a second CSP cycle by one or more intrinsic or non-CSP cardiac cycles. For example, during a capture threshold test or an electrode placement test, the VAT may be determined for one or more first CSP cardiac cycles when the CSP pulses are delivered at a first pacing pulse output (e.g., first pacing amplitude and pacing pulse width) at a first electrode location. The first CSP cycles may be followed by one or more intrinsic cardiac cycles or non-CSP cardiac cycles. VATs may subsequently be determined by processor 52 for one or more second CSP cardiac cycles when the CSP pulses are delivered at a second pacing pulse output different than the first pacing pulse output (e.g., a second higher or lower pacing amplitude and/or pacing pulse width) and/or at a second electrode location different than the first electrode location. Thus, “consecutively” determined CSP cardiac cycle VATs determined from CSP cardiac cycles used for detecting a sudden change in VAT may be separated by one or more cardiac cycles, which may be intrinsic cycles or paced cycles where pacing is delivered at a CSP site or a non-CSP site, e.g., the LV myocardium.

Processor 52 may return to block 156 to continue determining VATs for display in the GUI by display unit 54 when a change in VAT is not detected at block 160. When processor 52 detects a change in VAT at block 160, processor 52, in cooperation with display unit 54, may generate a notification or conspicuous indicator of the change in VAT in the GUI at block 162. The conspicuous indicator may include any of a change in color, size, font type, font style (e.g., bold, italicized, underlining etc.), background change, or other formatting of the displayed numeric value of the VAT. The conspicuous indicator may include a textual or written notification stating that the VAT change is detected, stating the VAT difference and/or other written notification relating to the detected VAT change and/or indicating a likely change in conduction system capture. The conspicuous indicator of the VAT change may additionally or alternatively include an audible notification. An audible notification may be a voiced notification, a beep, tone, change in the frequency or repetition rate of a beep or a tone, or other audible signal to notify a user of the detected VAT change.

The change in VAT may be a threshold increase or decrease in VAT. A decrease in VAT that is equal to or greater than the threshold difference may indicate capture of at least a portion of the conduction system. An increase in VAT greater than the threshold difference may indicate a loss of capture of the conduction system, which may be a total loss of capture by the CSP pulse or ventricular myocardial capture only without capture of the conduction system. When a decrease in VAT is detected, processor 52 may cause display unit 54 to change the display to provide a conspicuous indicator of the VAT change that indicates CSP capture, e.g., by changing the formatting of the VAT (shown as a numerical value) from red to green or another color or font or formatting change. When an increase in VAT is detected, processor 52 may cause display unit 54 to change the display to provide a conspicuous indicator of the VAT change that indicates a loss of CSP capture, e.g., changing the display of the VAT from green to red. While a color change is one way the VAT change may be conspicuously identified in the display at block 162, it is to be understood that other graphical, formatting or notification techniques may be used according to any of the examples listed herein, alone or in combination, with no limitation intended.

As long as processor 52 continues to receive input signals, processor 52 may continue to determine VATs and generate data for display in the GUI by display unit 54 by returning to block 156. A user may terminate the process of flow chart 150 at any time, e.g., by disconnecting ECG electrodes, entering an “end” command, logging out, powering down external device 50 or the like.

FIG. 8 is a conceptual diagram 200 of processing circuitry that may be included in external device 50 for obtaining differential unipolar and bipolar ECG signals according to some examples. The processing circuitry represented by the diagram of FIG. 8 may be implemented in hardware, firmware and/or software and may be included in processor 52 of external device 50 (see FIG. 1). In the example shown, the processing circuitry receives three, single-ended chest electrode inputs (C1, C2 and C3) and a reference electrode input. The reference electrode may be positioned at any location on the patient's body. The C1 electrode may represent a right chest electrode that may be placed at any medial or lateral location on or to the right of the sternum, posteriorly or anteriorly. The C1 electrode may be positioned between the second and sixth intercostal spaces on the right side of the patient, as examples. The C1 electrode can be the chest electrode of the three chest electrodes that is positioned nearest the right ventricle to obtain an ECG signal representative of or correlated to right ventricular electrical activity. In some examples, the C1 electrode can generally be positioned to obtain an ECG signal representative of global (right and left) ventricular activity.

The C2 electrode may represent a left chest electrode that may be placed at any medial or lateral location on or to the left of the sternum, posteriorly or anteriorly. The C2 electrode may be positioned between the third and seventh intercostal spaces on the left side of the patient, as examples. The C2 electrode can be the chest electrode of the three chest electrodes that is positioned nearest the left ventricle to obtain an ECG signal representative of or correlated to left ventricular electrical activity. In other examples, the C2 electrode can generally be positioned to obtain an ECG signal representative of global ventricular electrical activity.

The C3 electrode represents a third chest electrode that may be placed at any location, e.g., above the abdomen or last rib and below the neck or below the sternum, posteriorly or anteriorly, medially or laterally. In an example, the C3 electrode is positioned on the left chest at approximately the level of the first to fourth intercostal space, though other positions may be used. The three chest electrodes may be positioned in a triangular configuration generally centered over the heart or over the ventricles.

Any combination of the single-ended input signals may be used to determine a common mode signal that may represent a central terminal signal for computing a differential unipolar signal for at least one of the chest electrodes C1, C2 and/or C3. In one example, the four input signals from the C1, C2, C3 and reference electrodes are summed, as indicated by summation circuitry 202 for determining the common mode (CM) signal. In an example, summation circuitry 202 sums the C1, C2, C3 and reference input signals and performs a divide-by operation, e.g., a divide by two operation, to output the common mode (CM) signal 204.

A differential unipolar C1 signal 210 may be determined as the difference between the C1 input signal and the CM signal 204. A differential unipolar C2 signal 208 may be determined as the difference between the C2 input signal and the CM signal 204. A differential unipolar C3 signal 206 may be determined as the difference between the C3 input signal and the CM signal 204.

A first differential bipolar signal 216 may be determined as the difference of the unipolar C1 signal 210 and the unipolar C3 signal 206. A second differential bipolar signal 214 may be determined as the difference of the unipolar C1 signal 210 and the unipolar C2 signal 208. A third differential bipolar signal 212 may be determined as the difference of the unipolar C2 signal 208 and the unipolar C3 signal 206. Accordingly, in some examples, three chest electrodes and a reference electrode provide input signals to processing circuitry of external device 50 from which the processing circuitry may obtain up to three unipolar ECG signals (206, 208, and 210) and three bipolar ECG signals (212, 214 and 216). In other examples, at least one unipolar ECG signal, e.g., unipolar C2 signal 208, may be determined from the left chest electrode C2 input signal for use in determining VATs. In some examples, an additional unipolar ECG signal, e.g., unipolar C1 signal 210, may be determined using the right chest electrode C1 input signal

While up to three unipolar and optionally up to three bipolar ECG signals can be obtained by the processing circuitry represented in FIG. 8, it is recognized that a greater number of ECG signals could be obtained when additional chest electrodes are placed on the patient. However, the fewest number of electrodes may be used to simplify and shorten the set-up process required for processor 52 to obtain ECG signals from which CSP related data can be determined for presentation to a user in a GUI.

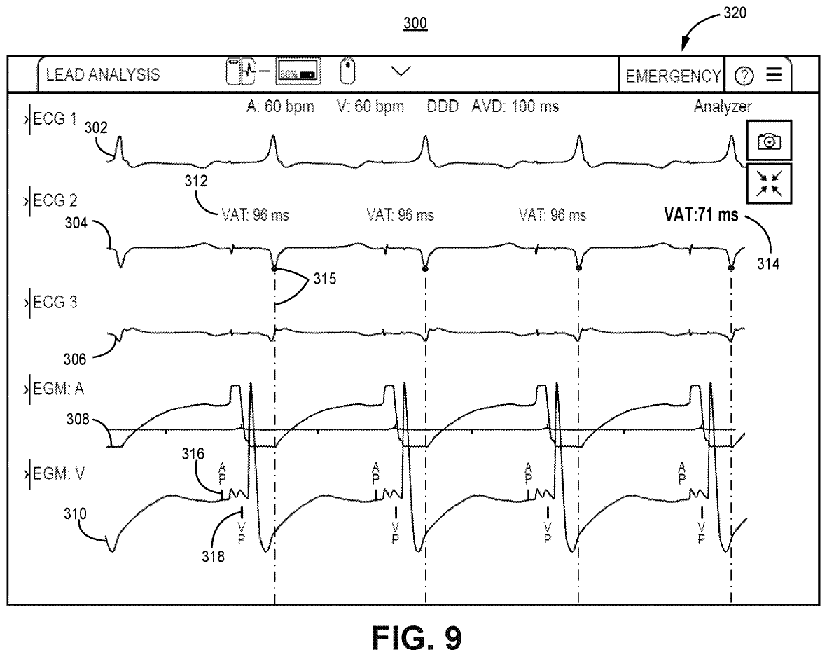

FIG. 9 is a conceptual diagram of a GUI 300 that may be displayed on display unit 54 of external device 50 according to one example. GUI 300 includes a display of three ECG signals 302, 304 and 306, an atrial (A) EGM signal 308 and a ventricular (V) EGM signal 310. Processor 52 may obtain the three ECG signals 302, 304 and 306 using the techniques described above in conjunction with FIG. 8. In the example of FIG. 9, processor 52 obtains at least three unipolar ECG signals from the input signals received from a reference electrode and three chest electrodes, which may be positioned as generally illustrated in FIG. 1. ECG 1 302 may represent the unipolar C1 ECG signal 210 shown in FIG. 8 that is obtained using the right chest electrode C1, e.g., the differential unipolar signal determined as the difference between a common mode (or central terminal) signal and the input signal from the C1 electrode.

ECG 2 304 may represent the unipolar C2 ECG signal 208 shown in FIG. 8 that is obtained using the left chest electrode C2, e.g., the differential unipolar signal determined as the difference between the common mode (or central terminal) signal and the input signal from the C2 electrode. ECG 3 306 may represent the unipolar C3 ECG signal 206 associated with the third chest electrode C3, e.g., the differential unipolar signal determined as the difference between the common mode (or central terminal signal) and the input signal from the C3 electrode. In other examples, any of the bipolar ECG signals described above in conjunction with FIG. 8 may be obtained by processor 52 and displayed, in addition to or alternatively to, the unipolar ECG signals shown in FIG. 9.

Furthermore, it is to be understood that while three ECG signals are shown displayed in the example of FIG. 9, in other examples none, one, two, or more than three ECG signals may be displayed in GUI 300.

In some examples, processor 52 obtains at least ECG 2 304 associated with a left chest electrode, e.g., the C2 electrode 42 shown in FIG. 1, for use in determining VATs. In some examples, processor 52 obtains at least ECG 1 302 associated with a right chest electrode, e.g., the C1 electrode 40 shown in FIG. 1, and at least ECG 2 304 associated with the left chest electrode and displays at least one of ECG 1 302 and/or ECG 2 304 in GUI 300. ECG 1 302 associated with the right chest electrode may be displayed to provide a visual representation to a user of the electrical activity of the right ventricle. The QRS waveforms of ECG 1 302 may present a RBB block appearance. In some patients, a pathologic RBB block condition may exist, which can be observed in ECG 1 302. In other instances, when the LBB is captured by CSP earlier than the depolarization of the RBB, a pacing-induced RBB block condition may exist, which can be observed in ECG 1 302 displayed in GUI 300.

ECG 2 304 associated with the left chest electrode may be displayed to provide a visual representation to a user of the electrical activity of the left ventricle. When CSP captures at least a portion of the conduction system, an evoked depolarization conducted via the LBB and subsequent LV myocardial depolarization can result in a relatively short VAT measured as the time interval from a delivered CSP pulse to a maximum (positive or negative) amplitude of ECG 2 304. Accordingly, processor 52 may obtain ECG 2 304 associated with the left chest electrode, determine VATs for each one of multiple paced cardiac cycles, and display the VATs 312. In other examples, a different fiducial point of the QRS waveform of the ECG signal may be used instead of a maximum amplitude, which may be an absolute maximum amplitude, for determining VATs. For example, processor 52 may identify a maximum positive slope, maximum negative slope, an amplitude threshold crossing (earliest or latest, positive-going or negative-going), return to baseline at the end of the QRS waveform or other fiducial QRS waveform point. Processor 52 and display unit 54 can be configured to cooperatively generate a display of the determined VATs for each of a plurality of pacing pulses. ECG 1 302 and/or ECG 2 304 may be displayed with the determined VATs. For example, multiple paced cardiac cycles of ECG 1 302 and/or ECG 2 304 may be displayed with each paced cardiac cycle annotated with the VAT 312 determined from the paced cardiac cycle.

The atrial EGM signal 308 and the ventricular EGM signal 310 may optionally be displayed in time alignment with the one or more ECG signals 302, 304 and 306. Each of the atrial EGM signal 308 and the ventricular EGM signal 310 may be transmitted by IMD 14 to external device 50, received by external device telemetry unit 58 and passed to display unit 54 for displaying in GUI 300.

GUI 300 may include pacing markers 316 and 318 indicating the timing of delivered atrial pacing pulses and ventricular pacing pulses, respectively, relative to the ECG and/or ECG signals displayed in GUI 300. In some examples, the ventricular pacing markers 318 may be shown in time alignment with each of multiple paced cardiac cycles of ECG 1 302 and/or ECG 2 304 and/or the associated VATs determined for each of the paced cardiac cycles. It is to be understood that ventricular pacing marker 318 can denote the timing of CSP pulses delivered to pace the ventricles via the conduction system. However, in some instances the ventricular pacing marker 318 may indicate the timing of a CSP pulse of a paced cardiac cycle that does not capture the conduction system. Accordingly, each ventricular pacing marker 318 may mark the timing of a CSP pulse that captures at least a portion of the conduction system, captures the ventricular myocardium only without capturing the conduction system, or fails to capture (loss of capture).

For instance, a ventricular pacing marker 318 may mark the timing of a CSP pulse that is delivered at a poor location for capturing the conduction system and/or is delivered at a pacing pulse energy that is less than a conduction system capture threshold. By displaying VATs in the GUI 300 that are formatted according to a value of the VAT and/or a relative difference from another VAT, as further described below, a user may readily identify and distinguish paced cardiac cycles that are associated with conduction system capture and paced cardiac cycles that are associated with loss of capture of the conduction system. In this way, the electrophysiological equipment, user expertise, time burden placed on the user in reviewing and analyzing ECG signals and potential for user error in selecting a CSP electrode implant site, CSP electrode vector, and pacing pulse output for capturing the conduction system can be minimized.

In the example of GUI 300, multiple paced cardiac cycles of at least one ECG signal are annotated with corresponding VATs 312. Processor 52 may be configured to compare a determined VAT to a threshold value. The threshold value may be defined based on an expected maximum VAT when a CSP pulse captures at least a portion of the conduction system. A VAT greater than the threshold value may indicate loss of CSP capture, even though ventricular myocardial capture without CSP capture may still be occurring. When the VAT is greater than the threshold value, display unit 54 may display the VAT according to a first format. When the VAT is less than the threshold value, display unit 54 may display the VAT according to a second format different than the first format. The first and second formats may include different font type, different font color and/or background color, different font size, different font style (e.g., bold, underlined, italicized etc.) or any combination thereof as examples.

In some examples, processor 52 may be configured to detect when a threshold difference is met by the difference (or ratio) between a first VAT determined for a first pacing pulse and a second VAT determined for a second pacing pulse. Display unit 54 may be configured to, in cooperation with processor 52, adjust the display of the determined VATs in response to the processor detecting the threshold difference between the first VAT and the second VAT by displaying the first VAT of the determined VATs according to a first format and displaying the second VAT of the determined VATs according to a second format different than the first format. Display unit 54, in cooperation with processor 52, can be configured to display the VATs determined for multiple paced cardiac cycles, determine when a threshold difference is met between two VATs, and display a conspicuous indicator in response to determining that the threshold difference is met. Display unit 54 may generate a display of a conspicuous indicator of the threshold difference being met by generating a visual notification, generating an audible notification, adjusting or changing a format of a displayed ventricular activation time to be different than a previously displayed ventricular activation time, and/or adjusting or changing a background of a displayed ventricular activation time. Display unit 54 may be configured to display, in a GUI, the activation times determined for multiple cardiac cycles and/or the activation time differences determined for multiple cardiac cycles, individually one cardiac cycle at a time or for multiple cardiac cycles simultaneously.

As shown in FIG. 9, the difference between the last VAT 314 and the preceding three VATs is greater than 20 ms. This change in VAT meeting a threshold difference may be detected by processor 52 such that the formatting of VAT 314 is adjusted by display unit 54 to be a larger and/or bolded font and/or may be displayed in a different font color, and/or different background as examples. For example, the VATs 312 that are greater than a threshold value of 90 ms, as an example, may be displayed in red font and the last VAT 314 that is less than the threshold value and/or is a threshold difference less than a preceding VAT may be displayed in a green font, bolded and/or enlarged font.

In some examples, an audible or text alert may be generated by display unit 54 to notify the user of the change in VAT. While a change from longer VATs 312 to a shorter VAT 314 is shown in FIG. 9, it is to be understood that the VATs may decrease or increase and may change back and forth between relatively longer and relatively shorter values during a CSP capture threshold test and/or positioning of a pacing electrode and/or testing of different pacing electrode vectors. Display unit 54, therefore, may adjust the display of VATs shown in GUI 300 to provide a conspicuous indicator of a threshold change in VAT multiple times based on comparisons to a threshold value and/or based on comparisons of a VAT difference to a threshold difference.

In some examples, the display unit 54 is further configured to display, with the determined VATs 312 and 314, the ECG signal used for determining the VATs, which is ECG 2 304 in the example shown, and a visual marker of at least one of the identified fiducial points of the ECG signal used to determine a VAT. In the example shown, the time interval from a delivered CSP pulse indicated by ventricular pacing marker 318 to the maximum peak amplitude (which is a negative polarity peak in ECG 2 304) is determined as the VAT 312. Display unit 54 may display one or more markers 315 marking the identified fiducial point used by processor 52 to determine the VAT. In the example shown, each paced cardiac cycle of ECG 2 304 is annotated with the VAT 312 or 314 determined for the corresponding paced cardiac cycle, and visual marker(s) 315 (shown as a circle and a vertical dashed line in FIG. 9) is/are displayed to mark each maximum peak of the pacing evoked QRS waveforms used in determining the VATs 312 and 314. In other examples, the visual markers may include a horizontal line or arrow extending from the ventricular pacing marker 318 to the time of the fiducial point and/or a symbol (e.g., a circle, triangle, square or other symbol) marking the fiducial point that is used to determine VAT relative to an immediately preceding CSP pulse.

GUI 300 may include various icons, menus, windows, patient information, IMD information etc., as generally shown by 320 in FIG. 9, which enable a user to identify the patient, the type of IMD implanted in the patient, the programmed pacing mode, atrial lower rate, ventricular lower rate, AV delay (AVD) or other device-or patient-related information. Various icons and menus may enable the user to switch between GUI screens or windows, change the speed of the scrolled ECG signal display, zoom in or out, freeze the display, store a screenshot of the display, print screen or perform other operations.

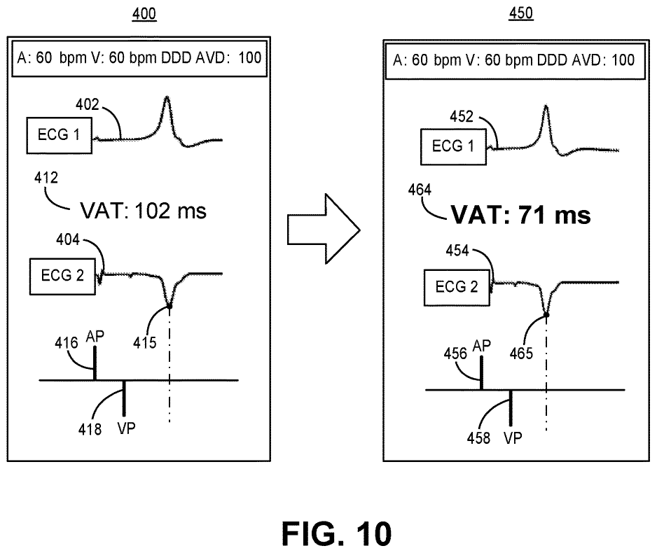

FIG. 10 is a conceptual diagram of two different screenshots 400 and 450 that may be displayed at different times in a GUI by display unit 54, in cooperation with processor 52, according to another example. In this example, processor 52 and display unit 54 are configured to obtain at least one ECG signal and display each one of a plurality of paced cardiac cycles one at a time. An individual paced cardiac cycles can be displayed in the GUI, e.g., as depicted by the image of screenshot 400 corresponding to one paced cardiac cycle followed by the image of screenshot 450 corresponding to a subsequent paced cardiac cycle. Each individual cardiac cycle displayed one at a time in the GUI can be annotated by the VAT determined for that paced cardiac cycle.

As shown in screenshot 400, the display unit 54 may display one single paced cardiac cycle of ECG 1 402 and/or ECG 2 404. ECG 1 402 may correspond to the unipolar CI ECG signal sensed using a right chest electrode as described above. ECG 2 404 may correspond to unipolar C2 ECG signal sensed using a left chest electrode as described above. In other examples, one or more bipolar ECG signals derived from the unipolar ECG signals obtained by processor 52 according to the techniques described in conjunction with FIG. 8 may be displayed in addition to or alternatively to the unipolar ECG signals 402 and 404. The single paced cardiac cycle of at least one ECG signal obtained by processor 52 may be displayed with pacing markers 416 and 418 indicating the timing of a delivered atrial pacing pulse and a ventricular pacing pulse, respectively, where the ventricular pacing marker 418 may represent a CSP pulse that may or may not capture the conduction system.

The VAT 412 determined for the paced cardiac cycle shown in screenshot 400 is displayed according to a first formatting scheme based on the value of the VAT 412 and/or the relative difference between the VAT 412 and another VAT determined for a different paced cardiac cycle. For instance, because the VAT 412 is greater than a threshold VAT, display unit 54 may display the VAT 412 in red font in an example. The font may be non-bolded, a relatively small size or otherwise conspicuously displayed to indicate that conduction system capture is unlikely. The threshold VAT may be 80 ms, 85 ms, 90 ms, 95 ms, or 100 ms as examples, with no limitation intended. The formatting of the VAT 412 indicates to a user that the value (102 ms in this illustrative example) indicates no capture (or loss of capture) of the conduction system. The formatting of the VAT 412 alone may indicate to a user that an adjustment to at least one of a higher pacing pulse output, different CSP electrode location and/or different CSP electrode vector is needed to achieve capture of the conduction system. In other examples, a text and/or audible notification may indicate that conduction system capture is not occurring. Other device-related information (e.g., atrial lower rate, ventricular lower rate, pacing mode, AV delay, remaining IMD battery life, etc.), patient-related data, and/or various icons, menus or other user interface features may be displayed in the GUI represented by screenshot 400.

Screenshot 450 represents the display of a different paced cardiac cycle of ECG 1 452 (corresponding to the same ECG signal as ECG 1 402 but a different paced cardiac cycle) and ECG 2 454 (corresponding to the same ECG signal as ECG 2 404 but a different paced cardiac cycle) than the paced cardiac cycle displayed in screenshot 400. In this case, the ventricular pacing marker 458 (delivered at an AV delay from atrial pacing marker 456) represents a CSP pulse that captures at least a portion of the conduction system. The displayed VAT 464 may be formatted according to a second formatting scheme to indicate that the VAT 464 represents capture of at least a portion of the conduction system. The VAT 464 may be displayed in a different color font (e.g., green font), different font style (e.g., bolded font), and/or larger font size than the display of VAT 412 to provide an adjusted or changed format of the displayed VAT as a conspicuous indicator to a user that the CSP pulse represented by ventricular pacing marker 458 likely captured at least a portion of the conduction system. The VAT 464 may be formatted differently than the formatting of VAT 412 in response to processor 52 determining that VAT 464 is less than a threshold VAT indicative of conduction system capture and/or that the difference between VAT 412 and VAT 464 is greater than a threshold difference. As generally described above in conjunction with FIG. 9, visual markers 415 and 465 may be displayed in the GUI to indicate to a user the fiducial point of the QRS waveform in the ECG signal 404 or 454 that is identified by processor 52 and used to calculate the VATs 412 and 464, respectively, relative to the respective ventricular pacing markers 418 and 458.

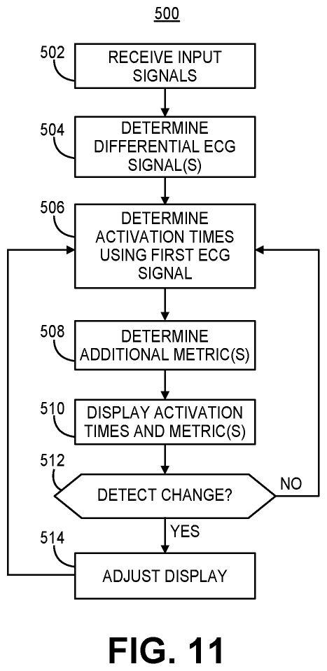

FIG. 11 is a flow chart 500 of a method for displaying data relating to CSP in a GUI by a medical device according to another example. At block 502, processor 52 receives input signals from multiple chest electrodes, e.g., at least one right chest electrode and at least one left chest electrode and a reference electrode, according to any of the examples described herein. In general, fewer than ten input signals are received as required for displaying a 12-lead ECG as commonly done during cardiac electrophysiology studies. As described in conjunction with FIG. 8, the input signals may consist of four single-end input signals received by processor 52 from three chest electrodes and one reference electrode so that processor 52 can obtain up to three unipolar ECG signals and/or up to three bipolar ECG signals at block 504 by determining a common mode (or central terminal) signal from the input signals and using the common mode (or central terminal) signal for deriving at least one differential unipolar ECG signal and, at least in some examples, one or more differential bipolar ECG signals.

At block 506, processor 52 determines VATs from a first one of multiple ECG signals obtained by processor 52. The ECG signal used at block 506 for determining VATs may be the unipolar C2 ECG signal determined as the difference of the input signal received from a left chest electrode (e.g., C2 electrode 42 shown in FIG. 1) and the central terminal signal or a bipolar ECG signal derived from the unipolar C2 ECG signal and another unipolar ECG signal. Processor 52 may determine the VAT for at least one paced cardiac cycle. In general, processor 52 determines the VAT for each paced cardiac cycle of two or more paced cardiac cycles.

The two or more paced cardiac cycles for which VATs are determined by processor 52 may include different pacing pulse energies of the delivered pacing pulses, e.g., during a CSP capture threshold test. The two or more paced cardiac cycles may include different pacing electrode sites as the CSP lead 18 (e.g., shown in FIG. 1) or the pacemaker 114 (e.g., shown in FIGS. 4 and 5) is being advanced to an implant site for delivering CSP, e.g., during an implant procedure. The two or more paced cardiac cycles may include different CSP electrode vectors, e.g., a unipolar pacing electrode vector, a bipolar pacing electrode vector, and/or bipolar pacing electrode vectors having reversed cathode and anode polarities. Different CSP electrode vectors may be tested during an implant procedure, CSP capture threshold test or other patient follow-up procedure. The two or more paced cardiac cycles, therefore, may each be paced according to different CSP control parameters in some instances to enable a user to identify CSP control parameters and/or a CSP electrode vector and/or CSP electrode site that successfully achieves CSP capture and improvement in ventricular electrical synchrony.

At block 508, processor 52 may determine one or more additional metrics for confirming CSP capture and/or a location of a CSP electrode. The one or more additional metrics may be determined from a second ECG signal obtained by processor 52 from a right chest electrode, e.g. the C1 electrode 40 illustrated in FIG. 1. The ECG signal used for determining a metric at block 508 may be a unipolar C1 ECG signal determined as the difference between the input signal from the C1 electrode and a central terminal signal or a bipolar ECG signal determined using the unipolar CI ECG signal and another unipolar ECG signal as generally described in conjunction with FIG. 8. One metric that may be determined at block 508 is a QRS width. The QRS width may be determined as the time interval from a delivered CSP pulse to a fiducial point of the QRS waveform. The fiducial point may approximate an ending time of the QRS waveform. Among the examples of fiducial points that may be identified by processor 52 and used to determine a QRS width are an amplitude threshold crossing, maximum negative slope, a dV/dt (or slope) threshold crossing, or an earliest baseline point of the ECG signal after a maximum peak of the QRS waveform (representing a return to baseline).

An additional or alternative metric that may be determined by processor 52 using a second ECG signal may be a QRS area of the second ECG signal following a delivered CSP pulse. A metric that may be determined by processor 52 using the second ECG signal may be a second activation time determined as the time interval from the CSP pulse to the maximum peak amplitude (or another fiducial point) of the QRS waveform of the second ECG signal. An additional or alternative metric that may be determined by processor 52 using a second ECG signal may be a peak time interval between a peak amplitude of the first ECG signal used to determine VAT and a peak amplitude of the second ECG signal following a delivered CSP pulse.

Other metrics determined at block 508 may include a QRS morphology change metric. A QRS morphology change metric may be determined from the ECG signal obtained by processor 52 using the right chest electrode, e.g., the unipolar CI ECG signal described above or a bipolar ECG signal obtained using the unipolar CI ECG signal. A QRS morphology change metric of an ECG signal obtained using a right chest electrode can indicate when RBB block occurs during LBB pacing and when a RBB block morphology due to LBB pacing is corrected. The QRS morphology change metric may be a ratio, difference or other quantitative relationship or relative comparison between absolute values of the peak amplitudes of a QRS waveform. For example, the QRS morphology metric may be a comparison of the R-wave peak amplitude to the Q-wave peak amplitude (absolute values). When a QR morphology is identified based on the R-wave peak (e.g., second peak of QRS waveform) being greater than the Q-wave peak (e.g., first peak of QRS waveform), a non-RBB block morphology may be detected by processor 52. When the second peak of the QRS waveform is less than the first peak, which may be associated with a Qr or QS morphology, a RBB block morphology may be detected by processor 52.

In other examples, the QRS morphology metric may be determined by processor 52 as a morphology matching score or waveform correlation metric determined between a QRS waveform and a previous QRS waveform or a previously established QRS waveform template, e.g., associated with a non-RBB block morphology. The QRS morphology metric may be determined using a Haar transform or other wavelet transform, for example. The QRS morphology metric may be determined beat-by-beat or from every nth paced cardiac cycle after a CSP pulse output and/or CSP electrode location is adjusted.

When the process of flow chart 500 is being performed during an implant procedure for positioning electrodes for CSP, e.g., in the area of the LBB, processor 52 may determine additional metric(s) at block 508 by detecting an LBB potential signal and/or injury current. Processor 52 may analyze the EGM signal received via lead 18 or transmitted from IMD 14 to detect an LBB potential signal for confirming a position of a CSP electrode, e.g., electrode 32 shown in FIG. 1, in the area of the LBB. The LBB potential signal is a signal spike occurring in the EGM signal immediately preceding a QRS signal attendant to the depolarization of the ventricular myocardium. The LBB potential signal represents the electrical potential conducted along the LBB that leads to ventricular myocardial depolarization.

In other examples, an injury current signal may be detected by processor 52 based on analysis of an EGM received by processor 52 during an intrinsic ventricular rhythm and sensed using a CSP electrode that is being positioned in the area of the LBB, for example. An elevated amplitude of the EGM signal immediately following LBB potential signal is evidence of injury current. An elevated amplitude following the LBB potential signal is therefore an indication that the CSP electrode is located in the left portion of the interventricular septum causing local injury near the LBB.

Processor 52 may detect the injury current by determining and storing a baseline amplitude (which may be an average baseline) of the EGM signal, detect the LBB potential signal and determine the maximum amplitude within an injury current detection window following the LBB potential signal. The injury current detection window can extend up to 10 ms, up to 20 ms, or up to 25 ms, as examples, after the LBB potential signal. Processor 52 may determine the difference between the baseline amplitude of the EGM signal (e.g., preceding the LBB potential signal) and the amplitude during the injury current detection window. When this amplitude difference of the EGM signal amplitude just prior to the LBB potential signal and just after the LBB potential signal is greater than a threshold amplitude difference or percentage change, e.g., at least 0.1 to 1 millivolt difference, an injury current may be detected by processor 52 indicating a LBB area position of the CSP electrode.

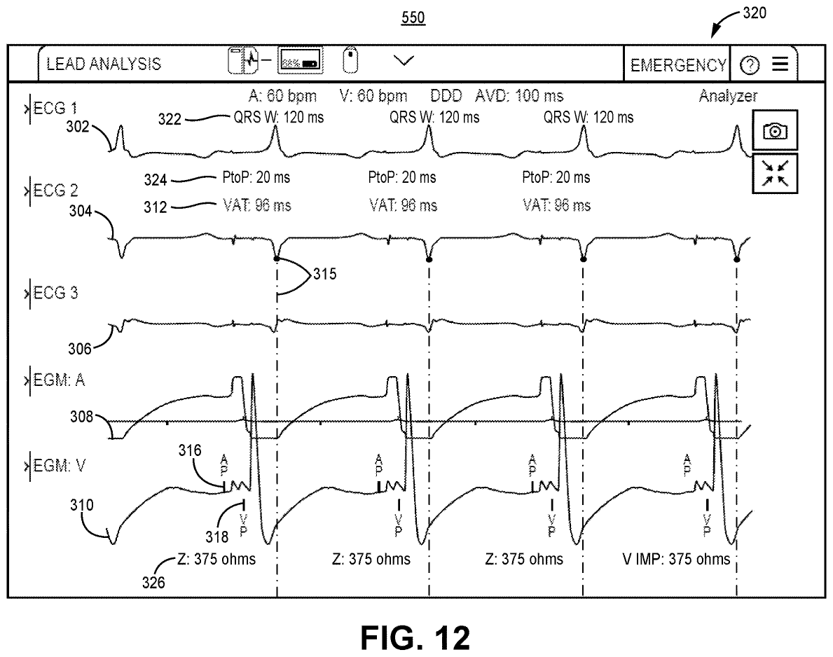

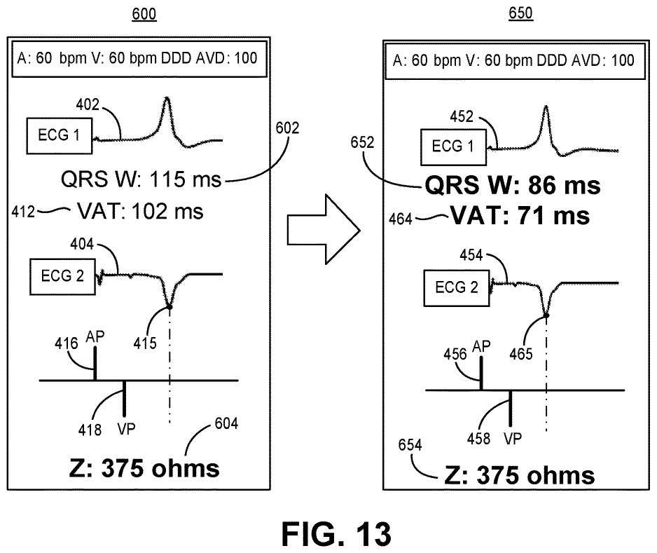

At block 510, display unit 54 may display in a GUI the determined VATs, differences between consecutively determined VATs, and/or one or more of the metrics determined at block 508 and/or changes in one or more of the metrics determined at block 508. Examples of GUIs that may include a display of a metric determined from a second ECG signal are shown in FIGS. 12 and 13 and described below. As generally described above, display unit 54 may display a conspicuous indicator in response to determining that a threshold difference between VATs is met at block 512. Display unit 54 may adjust the display of the VATs at block 514 in response to the processor 52 detecting a change in the determined VATs meeting a threshold difference (or being less than a threshold value) at block 512. The display of the VATs may be adjusted by changing the formatting of the displayed VATs, adding flashing text or icons, and/or adding a visual and/or audible notification of the detected VAT change. As further described below, in some examples, processor 52 may be configured to detect a change in a metric determined using a second ECG signal or EGM signal at block 512 and display a conspicuous indicator in response to detecting the change, e.g., by adjusting the display of the metric at block 514 by changing the formatting of the displayed metric and/or adding a visual or audible notification of the change in the metric. Any of the example metrics described herein may be displayed in conjunction with at least one ECG signal, the VATs (and/or determined VAT differences), CSP pulse markers, and optionally at least one EGM signal.

FIG. 12 is a diagram of a GUI 550 that may be cooperatively generated by processor 52 and display unit 54 for display on display unit 54 according to another example. Identical reference numbers shown in FIG. 12 correspond to like-numbered elements shown in FIG. 9 and described above. In the example of FIG. 12, display unit 54 may be configured to display one or more metrics determined using a second ECG signal obtained by processor 52. At least one ECG signal may be annotated with the VATs 312 as generally described above. A QRS width 322 determined by processor 52 from ECG 1 (or another non-displayed ECG signal obtained by processor 52) for at least one paced cardiac cycle may be displayed. Additionally or alternatively, a peak-to-peak time interval 324 determined by processor 52 using ECG 1 and ECG 2 (or another non-displayed ECG signal obtained by processor 52) may be displayed for at least one paced cardiac cycle. In the example shown, each cardiac cycle that follows a ventricular pacing marker 318 that represents a delivered CSP pulse may be annotated with a metric determined from a second ECG signal (other than the ECG signal used to determine the VAT), along with the determined VAT and/or a metric determined from the ventricular EGM signal 310 (e.g., LBB potential signal detection and/or injury current detection).

Additionally or alternatively, processor 52 may be configured to measure an impedance (Z) 326 using a CSP electrode, e.g., using pacing electrode 32 of CSP lead 18 or housing based electrode 102 of leadless pacemaker 114. The impedance 326 measured using a pacing electrode used to deliver CSP is also referred to herein as the “CSP impedance.” The CSP impedance 326 may be displayed on a beat-by-beat basis in GUI 550. In some instances, the impedance 326 may be measured by processor 52 while the pacing electrode 32 at the tip of CSP lead 18 is being advanced toward a CSP site during an implant procedure. A low impedance can indicate that the pacing electrode 32 at the tip of CSP lead 18 is in a blood pool and may need to be advanced further into tissue or may need to be retracted after being over-advanced, e.g., through interventricular septum. Accordingly, the impedance measured by processor 52 during delivery of an impedance measurement drive signal, e.g., which may be generated by pulse generator 60, may be displayed for one or more paced cardiac cycles in GUI 550.

When one or more additional metrics, such as the QRS width 322, QRS area (not shown in FIG. 12), QRS morphology metric (not shown in FIG. 12) and/or peak-to-peak time interval 324 or other metric(s), and/or impedance 326 are displayed in a GUI 550, display unit 54 may be configured to change or adjust the display in response to detecting a threshold change in the displayed metric or impedance. A displayed metric may be formatted according to a first formatting scheme when the metric is not indicative conduction system capture or not indicative of improvement in ventricular electrical synchrony (e.g., LBB capture but with pacing-induced RBB block as represented by a QRS width or peak-to-peak interval that is greater than a threshold value). The displayed metric may be formatted according to a second formatting scheme different than the first formatting scheme when the metric is indicative of conduction system capture with improved ventricular electrical synchrony. For example, when the peak-to-peak interval or the QRS width changes from being greater than to being less than a threshold value and/or represents a threshold change from a previous peak-to-peak interval or QRS width, respectively, the formatting of the peak-to-peak interval and/or QRS width may change in font, font color, font style, font size, background color, flashing text, or any combination thereof. It is to be understood that in addition to or instead of displaying the beat-to-beat values of a given metric, the beat-to-beat difference, percentage change or other relative change measurement of a given metric may be displayed in the GUI.

The impedance 326 may be displayed according to one formatting scheme when the impedance is lower than a threshold impedance indicative of the pacing electrode 32 (or pacemaker electrode 102) being in the blood pool. The impedance 326 may be displayed according to a second formatting scheme different than the first formatting scheme when the impedance is greater than the threshold impedance.

While QRS width and peak time intervals are shown in the GUI 550 as illustrative examples of additional metrics that may be determined and displayed in conjunction with VAT data and at least one ECG signal, it is to be understood that any of the example metrics described herein may be displayed alone or in any combination in any of the example GUIs shown and described herein in conjunction with at least one ECG signal and the VATs (and/or VAT differences). The VATs and/or VAT differences may be determined from an ECG signal obtained by processor 52 that is a different ECG signal than the displayed ECG signal. Among other metrics (or differences or changes thereof) that may be displayed are VATs determined from one or more additional ECG signal(s), QRS morphology metrics as described above in conjunction with FIG. 11, an indicator of LBB potential signal detection, and/or an indicator of injury current detection.

FIG. 13 is a conceptual diagram of two different screenshots 600 and 650 that may be displayed at different times in a GUI by display unit 54, in cooperation with processor 52, according to another example. Like-numbered elements in FIG. 13 correspond to identically numbered elements shown in FIG. 10 described above. In this example, processor 52 and display unit 54 are configured to determine and display a metric determined using a second ECG signal in addition to the VAT annotating each individual cardiac cycle displayed one at a time in the GUI. In the example shown, the QRS width 602 and QRS width 652 are shown for each of the two different cardiac cycles displayed in the individual screenshots 600 and 650.

The display of QRS width 652 may be adjusted or changed from the display of QRS width 602 when the QRS width 652 changes from being greater than a threshold width to less than a threshold width and/or when a difference between the two QRS widths 602 and 652 is greater than a threshold difference. A different formatting may be applied to QRS width 652 to provide a conspicuous indicator to a user of improved ventricular synchrony compared to the formatting applied to QRS width 602. In some instances, the VAT 464 may shorten when the LBB is captured by a CSP pulse but the QRS width determined from an ECG signal obtained using the right chest electrode (corresponding to right ventricular activation) may remain relatively long or even be increased due to a pacing-induced RBB block. The timing of a CSP pulse may be adjusted, e.g., by adjusting the AV delay, to shorten the QRS width. Accordingly, a change in the formatting applied to a displayed VAT 464 compared to the formatting applied to QRS width 652 (or another determined metric) may or may not occur on the same cardiac cycles. CSP control parameters may be adjusted by a user (or automatically by programming commands sent from external device 50 to IMD 14 or pacemaker 114) until both of the QRS width (or another metric) and the VAT are displayed according to a formatting scheme (e.g., green font, bolded, and/or enlarged font size as examples) that indicates CSP capture with improvement in ventricular electrical synchrony compared to the formatting scheme that indicates loss of capture of the conduction system and/or a lack of improvement in ventricular electrical synchrony.

A measured impedance 604 and 654 may optionally be shown in the GUI represented by screenshots 600 and 650. The measured impedance may indicate to a user when the CSP electrode is within cardiac tissue or in a blood pool. For example, in both screenshots 600 and 650, the impedance is relatively high, e.g., greater than 100 ohms or greater than 200 ohms, indicating that the CSP electrode is in cardiac tissue and not in the blood pool of a heart chamber. As such, the impedances 604 and 654 may be displayed according to a formatting scheme that indicates that the impedance is acceptable and that the CSP electrode is within cardiac tissue. For example, both of the impedances 605 and 654 may be shown in green, bolded font or according to another formatting scheme that indicates that the impedance is at an acceptable value. When the impedance is low, e.g., less than a threshold impedance corresponding to the CSP electrode being within the blood pool of a heart chamber, the impedance may be displayed according to a different formatting scheme, e.g., red font, to indicate that the impedance is not acceptable due to the CSP electrode likely being in a blood pool. An impedance displayed in a red font, for example, indicates to a user that the location of the pacing electrode needs to be adjusted.

In various examples, two or more parameters, e.g., VAT, QRS width, QRS area, peak-to-peak time interval, QRS morphology metric, LBB potential signal detection, injury current detection, and/or CSP impedance, may be displayed for each cardiac cycle of multiple paced cardiac cycles, shown individually one at a time or shown in groups of cardiac cycles, for example in a scrolling manner. The display of the cardiac cycles of one or more ECG signals annotated with VAT and, optionally, one or more metrics determined using a second ECG signal, a ventricular EGM signal and/or CSP impedance, may be toggled back and forth between the single cycle display as shown in FIGS. 10 and 13 and a scrolling or still multi-cycle display, e.g., as shown in FIGS. 9 and 12.

When two or more parameters are determined by processor 52 from ECG signals obtained by processor 52 and displayed, which may be with at least one of the obtained ECG signals, the formatting of the two or more parameters may be adjusted from one cardiac cycle to the next based on each individual value of the respective parameters.

Among the two or more parameters may be VAT, QRS width, QRS area, peak-to-peak time interval, QRS morphology matching score or other QRS morphology metric, LBB potential signal detection (or not), injury current detection (or not) and/or CSP impedance. Because all of the displayed parameters may not change relative to a threshold and/or relative to a previous value of the given parameter on the same cardiac cycle, the formatting for one parameter may remain according to a first formatting scheme while the formatting of a second parameter may be adjusted to a second formatting scheme based the individual values of the respective parameter. Processor 52 and display unit 54 may cooperatively adjust the format of each of the two or more parameters as each respective parameter is determined to change by a threshold difference and/or meet a threshold value.

Referring to FIG. 13 as an illustrative example, a first format of displayed parameter values may include a red font and a second format of displayed values may include a green font. The CSP impedances 604 and 654 may be displayed in green font to indicate the impedance values likely correspond to a tissue site of the CSP electrode, not a site within the blood pool of a vein or heart chamber. When the impedance is less than a threshold value, e.g., less than 100 ohms, the CSP impedance may be displayed in red, indicating that the CSP electrode should be further advanced, retracted, or otherwise adjusted. As seen in FIG. 13, the CSP impedance 605 may be displayed in green font in the image represented by screenshot 600. However, the QRS width 602 and the VAT 412 may both be displayed in red font in the screenshot 600 because both are greater than a respective threshold associated with CSP capture and improved ventricular electrical synchrony.

Referring to screenshot 650, display unit 54 may display the VAT 464 in green font when the VAT 464 falls below a threshold value and/or decreases by a threshold difference (or threshold percentage) from the VAT 412, shown in red font. Display unit 54 may display the QRS width 652 in green font when the QRS width 652 falls below a threshold value and/or decreases by a threshold difference (or threshold percentage) from the QRS width 602, shown in red font. An adjusted format is applied to both of the VAT 464 and QRS width 652 for the individual cardiac cycle displayed in screenshot 650. However, it is to be understood that in some cases in a given cardiac cycle the QRS width 652 may still be greater than a threshold value and still displayed in a first format, e.g., red font, while the VAT may have decreased to a value that results in an adjusted format, e.g., a change from a red font to a green font to provide a conspicuous indicator that CSP capture has occurred. A QRS width or other QRS morphology metric determined using an ECG signal obtained from the right chest electrode signal may remain displayed in the first format, e.g., red font, until further adjustments are made to the CSP pulse output, CSP electrode vector, CSP electrode location and/or CSP pulse timing (e.g., AV delay) and threshold change in the QRS width (or other metric) is detected indicating that CSP capture with improved ventricular electrical synchrony is achieved.

Thus, a combination of metrics or parameter values determined by processor 52 may be displayed in a combination of formats, depending on the value of each individual parameter. A user interacting with the GUI, may adjust one or more of a CSP electrode site, a CSP electrode vector, a CSP pulse output (e.g., pacing pulse amplitude and/or pulse width), a CSP time interval (e.g., AV delay), or other CSP control parameter until all displayed parameter values or metrics are presented according to a format, e.g., green font, that conspicuously indicates appropriate CSP electrode placement, CSP capture and improved ventricular electrical synchrony. In this way, the display of CSP related data and signals according to the techniques disclosed herein can simplify and expedite the process of evaluating CSP, selecting CSP control parameters, adjusting a CSP electrode site, selecting CSP electrode vector and verifying improved ventricular electrical synchrony during CSP.

In some examples, the VAT (and/or a VAT difference relative to a previous cardiac cycle) may be displayed in GUI 600 without additional metrics, such as the QRS width 602, until a threshold difference in VAT is detected. When the VAT is determined to be decreased by a threshold difference and/or to be less than a threshold VAT, a conspicuous indicator of the detected change in VAT may be displayed in the second screenshot 650 in combination with one or more additional metrics determined using another ECG signal and/or EGM signal. In this way, once CSP capture can be verified based on a decrease in VAT, determined from ECG 2 404/454 in this example, processor 52 and display unit 54 may be configured to cooperatively determine and display one or more additional metrics for verifying CSP capture without RBB block and/or appropriate CSP electrode placement. For example, the display of the QRS width 652, impedance 654, QRS area, peak time interval, a QRS morphology metric, LBB potential signal detection and/or injury current detection may be added (alone or in any combination) to the display of the VAT and/or VAT difference once the VAT change is detected. While not required in all examples, at least one ECG signal may be displayed with the determined metrics and optionally at least one EGM signal.

Accordingly, a first screen (e.g., screenshot 600) of a GUI displayed by display unit 54 may include a display of one or more VAT(s) that are greater than a threshold value, do not meet a threshold difference, and are not indicative of CSP capture. A second screen of the GUI (e.g., screenshot 650) may include a conspicuous indicator of a detected change in VAT(s) that meets a threshold difference and indicates CSP capture. The second screen may further include a display of one or more additional metrics determined using an ECG signal obtained by processor 52 (which may be a different ECG signal than the ECG signal used for determining the VAT(s) displayed in the first screen) and/or determined using a received EGM signal. The one or more additional metrics displayed in the second screen may or may not be displayed in the first screen. The one or more additional metrics may be displayed for confirming CSP capture, appropriate electrode location and/or a non-RBB block QRS morphology associated with CSP. It is to be understood that the “first” screen and the “second” screen of the GUI do not necessarily need to be discrete, separate screens. The first and second screens may be contiguous and may be displayed side by side. In other examples, the GUI may be displayed in a scrolling manner such that the first screen and the second screen may be in a continuous scrolling display of the signals and data where secondary metrics are added to the display of VATs when the VATs meet a threshold difference and/or are less than a threshold VAT.

It should be understood that, depending on the example, certain acts or events of any of the methods described herein can be performed in a different sequence, in parallel, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially. In addition, while certain aspects of this disclosure are described as being performed by a single processor, circuit or unit for purposes of clarity, it should be understood that the techniques of this disclosure may be performed by a combination of processors, units or circuits associated with, for example, a medical device system.

In one or more examples, the functions described may be implemented in hardware, software, firmware, or any combination thereof. If implemented in software, the functions may be stored as one or more instructions or code on a computer-readable medium and executed by one or more hardware-based processing units. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. Also, the techniques could be fully implemented in one or more circuits or logic elements.

Thus, a medical device system has been presented in the foregoing description with reference to specific examples. It is to be understood that various aspects disclosed herein may be combined in different combinations than the specific combinations presented in the accompanying drawings. It is appreciated that various modifications to the referenced examples may be made without departing from the scope of the disclosure and the following claims.

1. A medical device, comprising:
a processor configured to:
obtain one or more electrocardiogram (ECG) signals:
for each of a plurality of pacing pulses:
determine a ventricular activation time using a first ECG signal of the one or more ECG signals;
determine a difference between the ventricular activation time and a previously determined ventricular activation time; and
determine when the difference meets a threshold difference; and
a display unit in communication with the processor and configured to:
display at least one of the one or more ECG signals;
display at least one of the determined ventricular activation times or the determined differences; and
display a conspicuous indicator in response to determining that the threshold difference is met.

2. The medical device of claim 1, wherein the processor is further configured to obtain the first ECG signal by:
receiving four input signals by receiving one input signal from each of a reference electrode and three chest electrodes, the three chest electrodes including a left chest electrode and a right chest electrode;
determining a central terminal signal from the four input signals; and
determining a first unipolar ECG signal as a difference between the central terminal signal and the one of the four input signals that is received from the left chest electrode.

3. The medical device of claim 2, wherein the processor is further configured to obtain the first ECG signal by:
determining a second unipolar ECG signal from the input signals; and
determining the first ECG signal as a bipolar ECG signal by determining a difference between the first unipolar ECG signal and the second unipolar ECG signal.

4. The medical device of claim 2, wherein:
the processor is further configured to obtain the one or more ECG signals by determining a second unipolar ECG signal as a difference between an input signal from the right chest electrode and the central terminal signal; and
the display unit is further configured to display a plurality of cardiac cycles of the second unipolar ECG signal corresponding to at least a portion of the plurality of pacing pulses and aligned in time with respective ventricular activation times determined for at least the portion of the plurality of pacing pulses.

5. The medical device of claim 1, wherein the processor and the display unit are further configured to simultaneously display a plurality of cardiac cycles of at least one of the obtained ECG signal(s) and a plurality of the determined ventricular activation times, wherein the plurality of cardiac cycles of the at least one of the obtained ECG signal(s) and the plurality of ventricular activation times correspond to at least a portion of the plurality of pacing pulses.

6. The medical device of claim 1, wherein the processor and the display unit are further configured to consecutively display each one of a plurality of cardiac cycles of at least one of the obtained ECG signal(s), each one of the plurality of cardiac cycles being displayed individually one at a time with a respective determined ventricular activation time, the plurality of cardiac cycles corresponding to a least a portion of the plurality of pacing pulses.

7. The medical device of claim 1, wherein:
the processor is further configured to determine each of the ventricular activation times by:
identifying a fiducial point of the first ECG signal following a respective one of the plurality of pacing pulses; and
determining the ventricular activation time as a time interval from the respective one of the plurality of pacing pulses to the fiducial point; and
the display unit is further configured to display, with the determined ventricular activation times, the first ECG signal and a visual marker of at least one of the identified fiducial points of the first ECG signal.

8. The medical device of claim 1, wherein:
the processor is further configured to determine when a ventricular activation time of the determined ventricular activation times is greater than a threshold ventricular activation time; and
the display unit is further configured to display each of the ventricular activation times that are determined to be greater than the threshold ventricular activation time according to a first format and each of the ventricular activation times that are less than the threshold ventricular activation time according to a second format different than the first format.

9. The medical device of claim 1, wherein:
the processor is further configured to:
obtain the one or more ECG signals by receiving a plurality of input signals from at least a right chest electrode, a left chest electrode and a reference electrode, the one or more ECG signals comprising the first ECG signal associated with the left chest electrode and a second ECG signal associated with the right chest electrode;
for at least one of the plurality of pacing pulses, determine a QRS width using the second ECG signal; and
the display unit is further configured to display the determined QRS width.

10. The medical device of claim 1, wherein:
the processor is further configured to:
obtain the one or more ECG signals by receiving a plurality of input signals from at least a right chest electrode, a left chest electrode and a reference electrode, the plurality of ECG signals comprising the first ECG signal associated with the left chest electrode and a second ECG signal associated with the right chest electrode;
for at least one of the plurality of pacing pulses, determine at least one of a QRS area using the second ECG signal, a peak interval between a maximum peak of the first ECG signal and the second ECG signal, or a QRS morphology metric; and
the display unit is further configured to display at least one of the QRS area, peak interval or QRS morphology metric.

11. The medical device of claim 1, wherein the display unit is further configured to display the conspicuous indicator by at least one of: generating a visual notification, generating an audible notification, adjusting a format of the determined ventricular activation time to be different than a previously displayed ventricular activation time, and/or adjusting a background of the determined ventricular activation time.

12. The medical device of claim 1, further comprising a pulse generator configured to deliver the plurality of pacing pulses via a conduction system pacing electrode.

13. The medical device of claim 1, further comprising a telemetry circuit configured to receive a conduction system pacing pulse marker signal from an implantable medical device configured to deliver the plurality of pacing pulses as conduction system pacing pulses.

14.-20. canceled

21. A non-transitory computer readable medium storing a set of instructions that, when executed by processing circuitry of a medical device system, cause the medical device system to:
Obtain one or more electrocardiogram (ECG) signals;
for each of a plurality of pacing pulses:
determine a ventricular activation time using a first ECG signal of the one or more ECG signals;
determine a difference between the ventricular activation time and a previously determined ventricular activation time;
determine when the difference meets a threshold difference;
display at least one of the one or more ECG signals;
display at least one of the determined ventricular activation times or the determined differences; and
display a conspicuous indicator in response to determining that the threshold difference is met.

22. The non-transitory computer readable medium of claim 21 further comprising instructions that cause the medical device system to obtain the first ECG signal by:
receiving four input signals by receiving one input signal from each of a reference electrode and three chest electrodes, the three chest electrodes including a left chest electrode and a right chest electrode;
determining a central terminal signal from the four input signals; and
determining a first unipolar ECG signal as a difference between the central terminal signal and the one of the four input signals that is received from the left chest electrode.

23. The non-transitory computer readable medium of claim 22 further comprising instructions that cause the medical device system to obtain the first ECG signal by:
determining a second unipolar ECG signal from the input signals; and
determining the first ECG signal as a bipolar ECG signal by determining a difference between the first unipolar ECG signal and the second unipolar ECG signal.

24. The non-transitory computer readable medium of claim 22 further comprising instructions that cause the medical device system to:
obtain the one or more ECG signals by determining a second unipolar ECG signal as a difference between an input signal from the right chest electrode and the central terminal signal; and
display a plurality of cardiac cycles of the second unipolar ECG signal corresponding to at least a portion of the plurality of pacing pulses and aligned in time with respective ventricular activation times determined for at least the portion of the plurality of pacing pulses.

25. The non-transitory computer readable medium of claim 21 further comprising instructions that cause the medical device system to simultaneously display a plurality of cardiac cycles of at least one of the obtained ECG signal(s) and a plurality of the determined ventricular activation times, wherein the plurality of cardiac cycles of the at least one of the obtained ECG signal(s) and the plurality of ventricular activation times correspond to at least a portion of the plurality of pacing pulses.

26. The non-transitory computer readable medium of claim 21 further comprising instructions that cause the medical device system to consecutively display each one of a plurality of cardiac cycles of at least one of the obtained ECG signal(s), each one of the plurality of cardiac cycles being displayed individually one at a time with a respective determined ventricular activation time, the plurality of cardiac cycles corresponding to at least a portion of the plurality of pacing pulses.

27. The non-transitory computer readable medium of claim 21 further comprising instructions that cause the medical device system to:
determine each of the ventricular activation times by:
identifying a fiducial point of the first ECG signal following a respective one of the plurality of pacing pulses; and
determining the ventricular activation time as a time interval from the respective one of the plurality of pacing pulses to the fiducial point; and
display, with the determined ventricular activation times, the first ECG signal and a visual marker of at least one of the identified fiducial points of the first ECG signal.
